(19) United States
(12) Patent Application Publication
(10) Pub. No.: US 2023/0357110 A1
(43) Pub. Date: Nov. 9, 2023

(54) PROCESS

(71) Applicant: JOHNSON MATTHEY PUBLIC LIMITED COMPANY, London (GB)

(72) Inventors: Rowan BAILEY, Cambridge (GB); Chang GAO, Cambridge (GB); Damian GRAINGER, Cambridge (GB); Antonio ZANOTTI-GEROSA, Cambridge (GB)

(21) Appl. No.: 18/245,488

(22) PCT Filed: Nov. 3, 2021
(86) PCT No.: PCT/GB2021/052850

§ 371 Date: Mar. 15, 2023

(30) Foreign Application Priority Data
Nov. 4, 2020 (GB) 2017456.1

(51) Int. Cl. C07C 29/149

(52) U.S. Cl. CPC C07C 29/149

(57) ABSTRACT

The present invention is directed to a process for hydrogenation of an ester-containing substrate, comprising treating an ester-containing substrate with a base and a transition metal catalyst in the presence of molecular hydrogen, wherein the base is present in at least 30 mol % based upon the total amount of ester-containing substrate and wherein the substrate/catalyst loading is greater than or equal to 10,000/1.

### FIELD OF THE INVENTION

The present invention is directed to a process for the hydrogenation of an ester-containing substrate.

### BACKGROUND TO THE INVENTION

The reduction of esters is an essential transformation in the chemical industry as a route to primary alcohols. The reduction of esters has conventionally been carried out using reagents (in stoichiometric or excess quantities) such as sodium metal in ethanol (the Bouveault-Blanc reduction) or more recently with a metal hydride reagent, such as LiAlH₄ or NaBH₄. These reduction reactions are, however, difficult to carry out effectively on a large scale, not least due to safety concerns associated with an extremely exothermic quenching step. As such, research into the reduction of esters has more recently focussed on catalytic reduction using hydrogen gas. For example, Cu- or Zn-based heterogeneous catalysts are used forester reduction, primarily in the Natural Detergent Alcohol (NDA) market on very large scale. However, these methods require very high pressures and/or temperatures, in addition to large scale, dedicated production facilities. The chemoselectivity for ester reduction compared to other sensitive functional groups can also be problematic in some cases using these methods.

Although a number of other processes for ester hydrogenation using transition metal catalysts have been developed, these processes tend to require the use of large amounts of catalyst in order to achieve useful conversions and turnover numbers (TON). This is both expensive and has a negative environmental impact. Accordingly, there exists a need to provide an improved process for ester hydrogenation that requires a lower catalyst loading whilst still maintaining high catalyst activity and TON.

### SUMMARY OF THE INVENTION

The present invention provides an improved process for the hydrogenation of ester-containing compounds. The process is simple, economical, safe and can be operated in standard hydrogenation vessels. In certain embodiments, the process may have environmental benefits by requiring the use of much lower amounts of catalyst than is used in conventional processes.

In a first aspect, the present invention provides a process for hydrogenation of an ester-containing substrate, comprising treating an ester-containing substrate with a base and a transition metal catalyst in the presence of molecular hydrogen, wherein the base is present in at least 30 mol % based upon the total amount of ester-containing substrate and wherein the substrate/catalyst loading is greater than or equal to 10,000/1.

### Definitions

The point of attachment of a moiety or substituent is represented by “-”. For example, —OH is attached through the oxygen atom.

As used herein, the term “alkyl” refers to a straight-chain or branched saturated hydrocarbon group. In certain embodiments, the alkyl group may have from 1-20 carbon atoms, in certain embodiments from 1-15 carbon atoms, in certain embodiments, 1-8 carbon atoms. The alkyl group may be unsubstituted. Alternatively, the alkyl group may be substituted. Unless otherwise specified, the alkyl group may be attached at any suitable carbon atom and, if substituted, may be substituted at any suitable atom. Typical alkyl groups include but are not limited to methyl, ethyl, n-propyl, iso-propyl, n-butyl, iso-butyl, sec-butyl, tert-butyl, n-pentyl, n-hexyl and the like.

As used herein, the term “alkenyl” refers to a straight-chain or branched unsaturated hydrocarbon group comprising at least one carbon-carbon double bond.

As used herein, the term “alkynyl” refers to a straight-chain or branched unsaturated hydrocarbon group comprising at least one carbon-carbon triple bond.

As used herein, the term “cycloalkyl” is used to denote a saturated carbocyclic hydrocarbon radical. The cycloalkyl group may have a single ring or multiple condensed rings. In certain embodiments, the cycloalkyl group may have from 3-15 carbon atoms, in certain embodiments, from 3-10 carbon atoms, in certain embodiments, from 3-8 carbon atoms. The cycloalkyl group may be unsubstituted. Alternatively, the cycloalkyl group may be substituted. Unless other specified, the cycloalkyl group may be attached at any suitable carbon atom and, if substituted, may be substituted at any suitable atom. Typical cycloalkyl groups include but are not limited to cyclopropyl, cyclobutyl, cyclopentyl, cyclohexyl, adamantyl and the like.

As used herein, the term “cycloalkenyl” refers to an unsaturated, non-aromatic carbocyclic ring. The cycloalkenyl group therefore has at least one carbon-carbon double bond, but may have more. In certain embodiments, the cycloalkenyl group may have from 3-15 carbon atoms, in certain embodiments, from 3-10 carbon atoms, in certain embodiments, from 3-8 carbon atoms. The cycloalkenyl group may be unsubstituted. Alternatively, the cycloalkenyl group may be substituted. Unless other specified, the cycloalkenyl group may be attached at any suitable carbon atom and, if substituted, may be substituted at any suitable atom. Typical cycloalkenyl groups include but are not limited to cyclopropenyl, cyclobutenyl, cyclopentenyl, cyclohexenyl, and the like.

As used herein, the term “alkoxy” refers to an optionally substituted group of the formula alkyl-O- or cycloalkyl-O-, wherein alkyl and cycloalkyl are as defined above.

As used herein, the term “aryl” refers to an aromatic carbocyclic group. The aryl group may have a single ring or multiple condensed rings. In certain embodiments, the aryl group can have from 6-20 carbon atoms, in certain embodiments from 6-15 carbon atoms, in certain embodiments, 6-12 carbon atoms. The aryl group may be unsubstituted. Alternatively, the aryl group may be substituted. Unless otherwise specified, the aryl group may be attached at any suitable carbon atom and, if substituted, may be substituted at any suitable atom.

Examples of aryl groups include, but are not limited to, phenyl, naphthyl, anthracenyl and the like.

As used herein, the term “arylalkyl” refers to an optionally substituted group of the formula aryl-alkyl-, where aryl and alkyl are as defined above.

As used herein, the term “halogen”, “halo” or “hal” refers to —F, —Cl, —Br and —I.

As used herein, the term “heteroalkyl” refers to a straight-chain or branched saturated hydrocarbon group wherein one or more carbon atoms are independently replaced with one or more heteroatoms (e.g. nitrogen, oxygen, phosphorus and/or sulfur atoms). The heteroalkyl group may be unsubstituted. Alternatively, the heteroalkyl group may be substituted. Unless otherwise specified, the heteroalkyl group may be attached at any suitable atom and, if substituted, may be substituted at any suitable atom. Examples of heteroalkyl groups include but are not limited to ethers, thioethers, primary amines, secondary amines, tertiary amines and the like.

As used herein, the term “heterocycloalkyl” refers to a saturated cyclic hydrocarbon group wherein one or more carbon atoms are independently replaced with one or more heteroatoms (e.g. nitrogen, oxygen, phosphorus and/or sulfur atoms). The heterocycloalkyl group may be unsubstituted. Alternatively, the heterocycloalkyl group may be substituted.

Unless otherwise specified, the heterocycloalkyl group may be attached at any suitable atom and, if substituted, may be substituted at any suitable atom. Examples of heterocycloalkyl groups include but are not limited to epoxide, morpholinyl, piperadinyl, piperazinyl, thirranyl, pyrrolidinyl, pyrazolidinyl, imidazolidinyl, thiazolidinyl, thiomorpholinyl and the like.

As used herein, the term “heteroaryl” refers to an aromatic carbocyclic group wherein one or more carbon atoms are independently replaced with one or more heteroatoms (e.g. nitrogen, oxygen, phosphorus and/or sulfur atoms). The heteroaryl group may be unsubstituted. Alternatively, the heteroaryl group may be substituted. Unless otherwise specified, the heteroaryl group may be attached at any suitable atom and, if substituted, may be substituted at any suitable atom. Examples of heteroaryl groups include but are not limited to thienyl, furanyl, pyrrolyl, imidazolyl, pyrazolyl, thiazolyl, isothiazolyl, oxazolyl, isoxazolyl, triazolyl, tetrazolyl, thiadiazolyl, thiophenyl, oxadiazolyl, pyridinyl, pyrimidyl, benzoxazolyl, benzthiazolyl, benzimidazolyl, indolyl, quinolinyl and the like.

As used herein, the term “heterocycle” encompasses both heterocycloalkyl groups and heteroaryl groups.

As used herein, the term “substituted” refers to a group in which one or more hydrogen atoms are each independently replaced with substituents (e.g. 1, 2, 3, 4, 5 or more) which may be the same or different. Examples of substituents include but are not limited to -halo, —C(halo)₃, —R^{c}, ═O, ═S, —O—R^{c}, —S—R^{c}, —NR^{c}R^{d}, —CN, —NO₂, —C(O)—R^{c}, —COOR^{d}, —C(S)—R^{c}, —C(S)OR^{d}, —S(O)₂OH, —S(O)₂—R^{c}, —S(O)₂NR^{c}R^{d}, —O—S(O)—R^{c} and —CONR^{c}R^{d}, such as -halo, —C(halo)₃ (e.g. —CF₃), —R¹, —O—R^{c}, —NR^{c}R^{d}, —CN, or —NO₂. R^{c} and R^{d} are independently selected from the groups consisting of H, alkyl, aryl, arylalkyl, heteroalkyl, heteroaryl, or R^{c} and R^{d} together with the atom to which they are attached form a heterocycloalkyl group. R^{c} and R^{d} may be unsubstituted or further substituted as defined herein.

As used herein, the term “fatty acid” refers to a carboxylic acid with a long aliphatic chain (e.g. >6 carbon atoms), which can be either saturated or unsaturated. The aliphatic chain of the fatty acid may be branched or unbranched. In certain embodiments, the aliphatic chain of the fatty acid comprises 12 to 24 carbon atoms. In certain embodiments, the aliphatic chain of the fatty acid comprises 0 to 5 carbon-carbon double bonds.

As used herein, the term “fatty alcohol” refers to an alcohol with a long aliphatic chain (e.g. >6 carbon atoms), which can be either saturated or unsaturated. The aliphatic chain of the fatty alcohol may be branched or unbranched. In certain embodiments, the aliphatic chain of the fatty alcohol comprises 12 to 24 carbon atoms. In certain embodiments, the aliphatic chain of the fatty alcohol comprises 0 to 5 carbon-carbon double bonds.

As used herein, the term “wax ester” refers to an ester of a fatty acid and a fatty alcohol, wherein fatty acid and fatty alcohol are as defined above.

As used herein, the term “bidentate ligand” refers to a ligand that donates two pairs of electrons to a metal atom.

As used herein, the term “tridentate ligand” refers to a ligand that donates three pairs of electrons to a metal atom.

As used herein, the term “tetradentate ligand” refers to a ligand that donates four pairs of electrons to a metal atom.

As used herein, the term “Ru-SNS” refers to dichlorotriphenylphosphine[bis(2-(ethylthio)ethyl)amine]ruthenium(II).

As used herein, the term “Ru-PNN” refers to dichlorotriphenylphosphine[2-(diphenylphosphino)-N-(2-pyridinylmethyl)ethanamine]ruthenium(II).

As used herein, the term “Ru-SNN” refers to dichlorotriphenylphosphine[2-(ethylthio)-N-(2-pyridinylmethyl)ethanamine]ruthenium(II).

As used herein, the term “S/C” is an abbreviation for “substrate/catalyst” and is used to describe the catalyst loading employed in a reaction, i.e. it describes the molar ratio of ester-containing substrate and catalyst present in the reaction mixture. In the instance the ester-containing substrate contains more than one ester moiety, the S/C value is adjusted accordingly. For example, a molar ratio of triglyceride to catalyst of 10,000:1 equates to an S/C of 30,000:1 (as a triglyceride substrate contains three ester moieties).

As used herein, the term “turnover number” (TON) refers to the number of moles of substrate that a mole of catalyst can convert before becoming deactivated.

As used herein, unless otherwise specified, “mol %” describes the amount of moles of the specified material (e.g. a base) relative to the amount of moles of the ester-containing substrate, as a percentage. The “mol %” amount given for the specified material (e.g. a base) is the amount of that material employed in the reaction chamber (i.e. the location where the hydrogenation reaction takes place).

As used herein, the term “neat conditions” is used to describe a reaction which begins with a reaction mixture comprising at least 95% by volume of a mixture of ester-containing substrate and base.

As used herein, the term “hydrogenation” refers to hydrogenation using molecular hydrogen.

### DETAILED DESCRIPTION

Preferred and/or optional features of the invention will now be set out. Any aspect of the invention may be combined with any other aspect of the invention, unless the context demands otherwise. Any of the preferred or optional features of any aspect of the invention may be combined, singly or in combination, with any aspect of the invention, unless the context demands otherwise.

The present invention provides a process for hydrogenation of an ester-containing substrate, comprising treating an ester-containing substrate with a base and a transition metal catalyst in the presence of molecular hydrogen, wherein the base is present in at least 30 mol % based upon the total amount of ester-containing substrate and wherein the substrate/catalyst loading is greater than or equal to 10,000/1.

In preferred processes of the present invention, the base is present in at least 35 mol % based upon the total amount of ester-containing substrate, preferably in at least 40 mol % based upon the total amount of ester-containing substrate, more preferably in at least 45 mol % based upon the total amount of ester-containing substrate, and even more preferably in at least 50 mol % based upon the total amount of ester-containing substrate. Without wishing to be bound by theory, it is thought that the use of high amounts of base in the processes of the present invention allows for the use of lower catalyst loadings compared to known processes. In some cases, extremely low catalyst loadings have been achieved, e.g. S/C=greater than or equal to 100,000/1.

In preferred processes of the present invention, the base is present in less than or equal to 200 mol % based upon the total amount of ester-containing substrate, more preferably in less than or equal to 125 mol % based upon the total amount of ester-containing substrate.

In preferred processes of the present invention, the base is present in a range of 30 to 70 mol % based upon the total amount of ester-containing substrate, more preferably in a range of 30 to 60 mol % based upon the total amount of ester-containing substrate, even more preferably in a range of 30 to 50 mol % based upon the total amount of ester-containing substrate.

In preferred processes of the present invention, the base is a metal alkoxide. The metal alkoxide is preferably a metal methoxide, a metal ethoxide, a metal iso-propoxide, or a metal tert-butoxide. Preferred metal alkoxides include lithium ethoxide, sodium ethoxide, or potassium ethoxide.

In preferred processes of the present invention, the base is an alkali metal alkoxide. The alkali metal alkoxide is preferably an alkali metal methoxide, an alkali metal ethoxide, an alkali metal iso-propoxide, or an alkali metal tert-butoxide. The alkali metal alkoxide is more preferably an alkali metal methoxide or an alkali metal ethoxide.

In particularly preferred processes of the present invention, the base is an alkali metal ethoxide. The alkali metal ethoxide is preferably lithium ethoxide, sodium ethoxide or potassium ethoxide, more preferably sodium ethoxide.

In preferred processes of the present invention, the base is in solid form.

In preferred processes of the present invention, the base is supported. More preferably, the base is supported on a resin.

In preferred processes of the present invention, the process is carried out in the absence of solvent. This has the advantage of making the process both easier and less expensive to perform.

In alternative preferred processes of the present invention, the process is carried out under neat conditions.

In alternative preferred processes of the present invention, the process is carried out in the presence of at least one solvent.

Preferably, the at least one solvent is selected from an alcohol, toluene, THF and Me-THF. More preferably, the at least one solvent is selected from methanol, ethanol, toluene, THF and Me-THF. Most preferably, the at least one solvent is selected from methanol, ethanol, and toluene.

In preferred processes of the present invention, the at least one solvent is present in an amount of 10 to 100 vol % based upon the total volume of the ester-containing substrate, preferably 15 to 95 vol % based upon the total volume of the ester-containing substrate, more preferably 20 to 90 vol % based upon the total volume of the ester-containing substrate (e.g. 50 vol % based upon the total volume of the ester-containing substrate).

In preferred processes of the present invention, the volume ratio of the at least one solvent to the ester-containing substrate is less than or equal to 1:1, preferably less than or equal to 1:2.

In preferred processes of the present invention, the volume ratio of the at least one solvent to the ester-containing substrate is in the range 1:2 to 1:1, preferably in the range 1:2 to 1:1.5.

In preferred processes of the present invention, the process is carried out in the presence of more than one solvent. Preferred solvents are as described above.

In alternative preferred processes of the present invention, the process is carried out in the presence of a first solvent and a second solvent.

In preferred processes of the present invention, the first solvent is selected from toluene, THF and Me-THF. In preferred processes of the present invention, the second solvent is an alcohol, preferably ethanol.

In particularly preferred processes of the present invention, the first solvent is toluene and the second solvent is an alcohol, preferably ethanol.

In alternative particularly preferred processes of the present invention, the first solvent is THF and the second solvent is an alcohol, preferably ethanol.

In preferred processes of the present invention, the first solvent is present in an amount of 10 to 100 vol % based upon the total volume of the ester-containing substrate, preferably 15 to 95 vol % based upon the total volume of the ester-containing substrate, more preferably 20 to 90 vol % based upon the total volume of the ester-containing substrate (e.g. 50 vol % based upon the total volume of the ester-containing substrate).

In preferred processes of the present invention, the volume ratio of the first solvent to the ester-containing substrate is less than or equal to 1:1, preferably less than or equal to 1:2.

In preferred processes of the present invention, the volume ratio of the first solvent to the ester-containing substrate is in the range 1:2 to 1:1, preferably in the range 1:2 to 1:1.5.

In preferred processes of the present invention, the second solvent is present in an amount of 1 to 15 vol % based upon the total volume of ester-containing substrate, preferably in an amount of 1 to 10 vol % based upon the total volume of ester-containing substrate, preferably in an amount of 1 to 7.5 vol % based upon the total volume of the ester-containing substrate, more preferably in an amount of 1 to 5 vol % based upon the total volume of the ester-containing substrate.

In preferred processes of the present invention, the first solvent is present in an amount of 10 to 100 vol % based upon the total volume of the ester-containing substrate and the second solvent is present in an amount of 1 to 10 vol % based upon the total volume of ester-containing substrate, preferably the first solvent is present in an amount of 15 to 95 vol % based upon the total volume of the ester-containing substrate and the second solvent is present in an amount of 1 to 7.5 vol % based upon the total volume of the ester-containing substrate, more preferably the first solvent is present in an amount of 20 to 90 vol % based upon the total volume of the ester-containing substrate and the second solvent is present in an amount of 1 to 5 vol % based upon the total volume of the ester-containing substrate.

In preferred processes of the present invention, the process is conducted at a temperature in the range 20 to 150° C., more preferably in the range 20 to 140° C., more preferably in the range 25 to 130° C., more preferably in the range 25 to 120° C., more preferably in the range 30 to 100° C., more preferably in the range 30 to 90° C., more preferably in the range 30 to 80° C., more preferably in the range 35 to 75° C., even more preferably in the range 37.5 to 60° C., even more preferably in the range 40 to 55° C., and most preferably in the range 40 to 50° C. (e.g. 40° C.). The preferred processes of the present invention are conducted at relatively low temperatures, meaning that the processes are more economical because the energy input to the reaction is lower. It is thought that lower temperatures for the ester hydrogenation may also help to improve catalyst stability.

Preferred processes of the present invention are conducted at a pressure that is at least 5 bar, more preferably at least 10 bar, even more preferably at least 20 bar, even more preferably at least 30 bar, even more preferably at least 40 bar, and most preferably at least 50 bar.

Preferred processes of the present invention are conducted at a pressure that is in the range 5 to 100 bar, more preferably in the range 10 to 95 bar, even more preferably in the range 20 to 90 bar, even more preferably in the range 25 to 70 bar, and most preferably in the range 30 to 50 bar.

Preferred processes of the present invention are conducted for a duration of 1 to 24 hours, more preferably 2 to 16 hours, even more preferably 3 to 10 hours, and most preferably 4 to 8 hours.

The processes of the present invention require a low catalyst loading, whilst still achieving an industrially useful TON and conversion for the ester hydrogenation. A lower catalyst loading means that the reactions are greener and more efficient. A lower catalyst loading also means that the cost of the reaction can be reduced.

In the process of the present invention, the substrate/catalyst loading is greater than or equal to 10,000/1. In preferred processes of the present invention, the substrate/catalyst loading is greater than or equal to 20,000/1, even more preferably greater than or equal to 30,000/1, even more preferably greater than or equal to 40,000/1, even more preferably greater than or equal to 50,000/1, even more preferably greater than or equal to 60,000/1, even more preferably greater than or equal to 70,000/1, even more preferably greater than or equal to 80,000/1, even more preferably greater than or equal to 90,000/1, even more preferably greater than or equal to 100,000/1, even more preferably greater than or equal to 200,000/1, even more preferably greater than or equal to 300,000/1, even more preferably greater than or equal to 400,000/1, even more preferably greater than or equal to 500,000/1, even more preferably greater than or equal to 600,000/1, even more preferably greater than or equal to 700,000/1, even more preferably greater than or equal to 800,000/1, even more preferably greater than or equal to 900,000/1, even more preferably greater than or equal to 1,000,000/1.

In some embodiments of the process of the present invention, the substrate/catalyst loading is less than or equal to 2,000,000/1.

The process of the present invention employs a transition metal catalyst. The transition metal catalyst may be pre-formed or may be formed in situ during the ester hydrogenation reaction. Preferably, the transition metal catalyst is pre-formed. Alternatively, the transition metal catalyst is formed in situ during the ester hydrogenation reaction.

In preferred processes of the present invention, the transition metal in the transition metal catalyst is a Group 6, Group 7, Group 8, or Group 9 transition metal. More preferably, the transition metal in the transition metal catalyst is a Group 7, Group 8, or Group 9 transition metal. Even more preferably, the transition metal in the transition metal catalyst is a Group 8 transition metal.

In preferred processes of the present invention, the transition metal in the transition metal catalyst is selected from Mo, Mn, Fe, Ru, Co and Os. More preferably, the transition metal in the transition metal catalyst is selected from Ru and Os. Most preferably, the transition metal in the transition metal catalyst is Ru.

In preferred processes of the present invention, the transition metal catalyst employed in the process of the present invention comprises a tridentate ligand.

In preferred processes of the present invention, the transition metal catalyst comprises a tridentate ligand having a Formula (I)

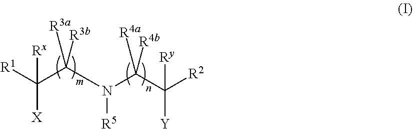

wherein:
X is selected from —SR^{a}, —OR^{a}, —CR^{a}, —NR^{a}R^{b}, —PR^{a}R^{b}, —P(═O)R^{a}R^{b}, —OPR^{a}R^{b}, and —NHPR^{a}R^{b};
R¹ and R^{x} are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl, or R¹ and one of R^{3a} and R^{3b} or R^{x} and one of R^{3a} and R^{3b} together with the atoms to which they are bound, form a ring;
or X is a heteroatom and when taken together with R¹ it forms an optionally substituted heterocycle when R^{x} is absent;
Y is selected from —SR^{a}, —OR^{a}, —CR^{a}, —NR^{a}R^{b}, —PR^{a}R^{b}, —P(═O)R^{a}R^{b}, —OPR^{a}R^{b}, and —NHPR^{a}R^{b};
R² and R^{y} are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl, or R² and one of R^{4a} and R^{4b} or R^{y} and one of R^{4a} and R^{4b} together with the atoms to which they are bound, form a ring;
or Y is a heteroatom and when taken together with R² it forms an optionally substituted heterocycle when R^{y} is absent;
R^{3a}, R^{3b}, R^{4a}, and R^{4b} are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl, or R^{3a} and one of R^{4a} and R^{4b} or R^{3b} and one of R^{4a} and R^{4b} together with the atoms to which they are bound, form a heterocycle;
R⁵ is selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl;
each m and n is independently 1 or 2; and
R^{a} and R^{b}, if present, are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₅₋₂₀-aryl, and substituted or unsubstituted C₄₋₂₀-heteroaryl; or when X and/or Y is —NR^{a}R^{b}, —PR^{a}R^{b}, —OPR^{a}R^{b}, or —NHPR^{a}R^{b}, R^{a} and R^{b} together with the heteroatom to which they are attached form a heterocycle.

In tridentate ligands of Formula (I), X is preferably selected from —SR^{a}, —CR^{a}, —NR^{a}R^{b}, —PR^{a}R^{b}, and —NHPR^{a}R^{b}. More preferably, X is selected from —SR^{a}, —PR^{a}R^{b}, and —NHPR^{a}R^{b}. Even more preferably, X is selected from —SR^{a} and —PR^{a}R^{b}. Most preferably, X is —SR^{a}.

In tridentate ligands of Formula (I), R¹ and R^{x} are each independently preferably selected from hydrogen, substituted or unsubstituted C₁₋₂₀-alkyl, substituted or unsubstituted C₁₋₂₀-heteroalkyl and substituted or unsubstituted C₃₋₂₀-cycloalkyl. More preferably, R¹ and R^{x} are each independently selected from hydrogen and substituted or unsubstituted C₁₋₂₀-alkyl. Even more preferably, R¹ and R^{x} are each hydrogen.

In alternative preferred tridentate ligands of Formula (I), X is a heteroatom and when taken together with R¹ it forms an optionally substituted heterocycle when R^{x} is absent. More preferably, X is a heteroatom and when taken together with R¹ it forms an optionally substituted heteroaromatic ring when R^{x} is absent. More preferably, the optionally substituted heteroaromatic ring is an optionally substituted nitrogen-containing heteroaromatic ring. Even more preferably, the optionally substituted nitrogen-containing heteroaromatic ring is selected from pyridinyl, pyrrolyl, imidazolyl, pyrazolyl, thiazolyl, isothiazolyl, oxazolyl, isoxazolyl, triazolyl, tetrazolyl, thiadiazolyl, oxadiazolyl, pyrimidyl, benzoxazolyl, benzthiazolyl, benzimidazolyl, indolyl, and quinolinyl. Even more preferably, the optionally substituted nitrogen-containing heteroaromatic ring is selected from pyridinyl, pyrrolyl, imidazolyl, pyrazolyl, triazolyl, tetrazolyl, and pyrimidyl. Most preferably, the optionally substituted nitrogen-containing heteroaromatic ring is pyridinyl.

In tridentate ligands of Formula (I), Y is preferably selected from —SR^{a}, —CR^{a}, —NR^{a}R^{b}, —PR^{a}R^{b}, and —NHPR^{a}R^{b}. More preferably, Y is selected from —SR^{a}, —PR^{a}R^{b}, and —NHPR^{a}R^{b}. Even more preferably, Y is selected from —SR^{a} and —PR^{a}R^{b}. Most preferably, Y is —SR^{a}.

In tridentate ligands of Formula (I), R² and R^{y} are each independently preferably selected from hydrogen, substituted or unsubstituted C₁₋₂₀-alkyl, substituted or unsubstituted C₁₋₂₀-heteroalkyl and substituted or unsubstituted C₃₋₂₀-cycloalkyl. More preferably, R² and R^{y} are each independently selected from hydrogen and substituted or unsubstituted C₁₋₂₀-alkyl. Even more preferably, R² and R^{y} are each hydrogen.

In alternative preferred tridentate ligands of Formula (I), Y is a heteroatom and when taken together with R² it forms an optionally substituted heterocycle when R^{y} is absent. More preferably, Y is a heteroatom and when taken together with R² it forms an optionally substituted heteroaromatic ring when R^{y} is absent. More preferably, the optionally substituted heteroaromatic ring is an optionally substituted nitrogen-containing heteroaromatic ring. Even more preferably, the optionally substituted nitrogen-containing heteroaromatic ring is selected from pyridinyl, pyrrolyl, imidazolyl, pyrazolyl, thiazolyl, isothiazolyl, oxazolyl, isoxazolyl, triazolyl, tetrazolyl, thiadiazolyl, oxadiazolyl, pyrimidyl, benzoxazolyl, benzthiazolyl, benzimidazolyl, indolyl, and quinolinyl. Even more preferably, the optionally substituted nitrogen-containing heteroaromatic ring is selected from pyridinyl, pyrrolyl, imidazolyl, pyrazolyl, triazolyl, tetrazolyl, and pyrimidyl. Most preferably, the optionally substituted nitrogen-containing heteroaromatic ring is pyridinyl.

In tridentate ligands of Formula (I), R^{3a}, R^{3b}, R^{4a}, and R^{4b} are each independently preferably selected from hydrogen, substituted or unsubstituted C₁₋₂₀-alkyl, substituted or unsubstituted C₁₋₂₀-heteroalkyl and substituted or unsubstituted C₃₋₂₀-cycloalkyl. More preferably, R^{3a}, R^{3b}, R^{4a}, and R^{4b} are each independently selected from hydrogen and substituted or unsubstituted C₁₋₂₀-alkyl. Even more preferably, R^{3a}, R^{3b}, R^{4a}, and R^{4b} are each hydrogen.

In alternative preferred tridentate ligands of Formula (I), R^{3a} and one of R^{4a} and R^{4b} or R^{3b} and one of R^{4a} and R^{4b} together with the atoms to which they are bound, form a heterocycle. Preferably, the heterocycle is a six-membered ring heterocycle.

In tridentate ligands of Formula (I), R⁵ is preferably selected from hydrogen, substituted or unsubstituted C₁₋₂₀-alkyl, substituted or unsubstituted C₁₋₂₀-heteroalkyl and substituted or unsubstituted C₃₋₂₀-cycloalkyl. More preferably, R⁵ is selected from hydrogen and substituted or unsubstituted C₁₋₂₀-alkyl. Even more preferably, R⁵ is hydrogen.

In tridentate ligands of Formula (I), each m and n is preferably 1.

In tridentate ligands of Formula (I), R^{a} and R^{b}, if present, are each independently preferably selected from hydrogen, substituted or unsubstituted C₁₋₂₀-alkyl, substituted or unsubstituted C₁₋₂₀-heteroalkyl, substituted or unsubstituted C₃₋₂₀-cycloalkyl, substituted or unsubstituted C₆₋₂₀-aryl, and substituted or unsubstituted C₄₋₂₀-heteroaryl. More preferably, R^{a} and R^{b}, if present, are each independently selected from hydrogen, substituted or unsubstituted C₁₋₂₀-alkyl (e.g. C₁₋₁₀-alkyl) and substituted or unsubstituted C₆₋₂₀-aryl. Particularly preferred C₁₋₂₀-alkyl groups include ethyl, ethyl, n-propyl, iso-propyl, n-butyl, sec-butyl, tert-butyl, pentyl, and hexyl, more preferably methyl, ethyl, iso-propyl, tert-butyl, even more preferably ethyl. Preferred C₆₋₂₀-aryl groups include phenyl, tolyl, xylyl, and methoxyphenyl, more preferably phenyl.

In alternative preferred tridentate ligands of Formula (I), when X and/or Y is —NR^{a}R^{b}, —PR^{a}R^{b}, —OPR^{a}R^{b}, or —NHPR^{a}R^{b}, R^{a} and R^{b} together with the heteroatom to which they are attached form a heterocycle.

In preferred processes of the present invention, the transition metal catalyst comprises a tridentate ligand having a Formula (I) wherein:
X is selected from —SR^{a}, —CR^{a}, —NR^{a}R^{b}, —PR^{a}R^{b}, and —NHPR^{a}R^{b};
R¹ and R^{x} are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl;
or X is a heteroatom and when taken together with R¹ it forms an optionally substituted heteroaromatic ring when R^{x} is absent, wherein the heteroaromatic ring is a nitrogen-containing heteroaromatic ring;
Y is selected from —SR^{a}, —CR^{a}, —NR^{a}R^{b}, —PR^{a}R^{b}, and —NHPR^{a}R^{b};
R² and R^{y} are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl;
or Y is a heteroatom and when taken together with R² it forms an optionally substituted heteroaromatic ring when R^{y} is absent, wherein the heteroaromatic ring is a nitrogen-containing heteroaromatic ring;
R^{3a}, R^{3b}, R^{4a}, R^{4b} and R⁵ are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl; each m and n is independently 1 or 2; and
R^{a} and R^{b}, if present, are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₅₋₂₀-aryl, and substituted or unsubstituted C₄₋₂₀-heteroaryl; or when X and/or Y is —NR^{a}R^{b}, —PR^{a}R^{b} or —NHPR^{a}R^{b}, R^{a} and R^{b} together with the heteroatom to which they are attached form a heterocycle.

In preferred processes of the present invention, the transition metal catalyst comprises a tridentate ligand having a Formula (I) wherein:
X is selected from —SR^{a}, —PR^{a}R^{b}, and —NHPR^{a}R^{b};
R¹ and R^{x} are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl;
or X is a heteroatom and when taken together with R¹ it forms an optionally substituted heteroaromatic ring when R^{x} is absent, wherein the heteroaromatic ring is a nitrogen-containing heteroaromatic ring selected from pyridinyl, pyrrolyl, imidazolyl, pyrazolyl, thiazolyl, isothiazolyl, oxazolyl, isoxazolyl, triazolyl, tetrazolyl, thiadiazolyl, oxadiazolyl, pyrimidyl, benzoxazolyl, benzthiazolyl, benzimidazolyl, indolyl, and quinolinyl;
Y is selected from —SR^{a}, —PR^{a}R^{b}, and —NHPR^{a}R^{b};
R² and R^{y} are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl;
or Y is a heteroatom and when taken together with R² it forms an optionally substituted heteroaromatic ring when R^{y} is absent, wherein the heteroaromatic ring is a nitrogen-containing heteroaromatic ring selected from pyridinyl, pyrrolyl, imidazolyl, pyrazolyl, thiazolyl, isothiazolyl, oxazolyl, isoxazolyl, triazolyl, tetrazolyl, thiadiazolyl, oxadiazolyl, pyrimidyl, benzoxazolyl, benzthiazolyl, benzimidazolyl, indolyl, and quinolinyl;
R³, R^{3b}, R^{4a}, R^{4b} and R⁵ are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl;
each m and n is independently 1 or 2; and
R^{a} and R^{b}, if present, are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₅₋₂₀-aryl, and substituted or unsubstituted C₄₋₂₀-heteroaryl; or when X and/or Y is —PR^{a}R^{b} or —NHPR^{a}R^{b}, R^{a} and R^{b} together with the heteroatom to which they are attached form a heterocycle.

In preferred processes of the present invention, the transition metal catalyst comprises a tridentate ligand having a Formula (I)
wherein:
X is selected from —SR^{a} and —PR^{a}R^{b};
R¹ and R^{x} are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl;
or X is a heteroatom and when taken together with R¹ it forms an optionally substituted heteroaromatic ring when R^{x} is absent, wherein the heteroaromatic ring is a nitrogen-containing heteroaromatic ring selected from pyridinyl, pyrrolyl, imidazolyl, pyrazolyl, triazolyl, tetrazolyl, and pyrimidyl;
Y is selected from —SR^{a} and —PR^{a}R^{b};
R² and R^{y} are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl;
or Y is a heteroatom and when taken together with R² it forms an optionally substituted heteroaromatic ring when R^{y} is absent, wherein the heteroaromatic ring is a nitrogen-containing heteroaromatic ring selected from pyridinyl, pyrrolyl, imidazolyl, pyrazolyl, triazolyl, tetrazolyl, and pyrimidyl;
R³, R^{3b}, R^{4a}, R^{4b} and R⁵ are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₅₋₂₀-aryl, and substituted or unsubstituted C₄₋₂₀-heteroaryl; each m and n is independently 1 or 2; and
R^{a} and R^{b}, if present, are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₅₋₂₀-aryl, and substituted or unsubstituted C₄₋₂₀-heteroaryl; or when X and/or Y is —PR^{a}R^{b}, R^{a} and R^{b} together with the heteroatom to which they are attached form a heterocycle.

In preferred processes of the present invention, the transition metal catalyst comprises a tridentate ligand having a Formula (I), wherein:
X is —SR^{a};
R¹ and R^{x} are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl;
Y is —SR^{a};
R² and R^{y} are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl;
R^{3a}, R^{3b}, R^{4a}, R^{4b} and R⁵ are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl; each m and n is independently 1 or 2; and
R^{a} are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₅₋₂₀-aryl, and substituted or unsubstituted C₄₋₂₀-heteroaryl.

Preferably, the transition metal catalyst comprises a tridentate ligand having a Formula (I), wherein:
X is —SR^{a};
R¹ and R^{x} are each independently selected from hydrogen, substituted or unsubstituted C₁₋₂₀-alkyl, substituted or unsubstituted C₁₋₂₀-heteroalkyl, and substituted or unsubstituted C₃₋₂₀-cycloalkyl;
Y is —SR^{a};
R² and R^{y} are each independently selected from hydrogen, substituted or unsubstituted C₁₋₂₀-alkyl, substituted or unsubstituted C₁₋₂₀-heteroalkyl, and substituted or unsubstituted C₃₋₂₀-cycloalkyl;
R^{3a}, R^{3b}, R^{4a}, R^{4b} and R⁵ are each independently selected from hydrogen, substituted or unsubstituted C₁₋₂₀-alkyl, substituted or unsubstituted C₁₋₂₀-heteroalkyl, and substituted or unsubstituted C₃₋₂₀-cycloalkyl;
each m and n is independently 1 or 2; and R^{a} are each independently selected from hydrogen, substituted or unsubstituted C₁₋₂₀-alkyl, substituted or unsubstituted C₁₋₂₀-heteroalkyl, and substituted or unsubstituted C₃₋₂₀-cycloalkyl.

More preferably, the transition metal catalyst comprises a tridentate ligand having a Formula (I), wherein:
X is —SR^{a};
R¹ and R^{x} are each independently selected from hydrogen and substituted or unsubstituted C₁₋₂₀-alkyl;
Y is —SR^{a};
R² and R^{y} are each independently selected from hydrogen and substituted or unsubstituted C₁₋₂₀-alkyl;
R³, R^{3b}, R^{4a}, R^{4b} and R⁵ are each independently selected from hydrogen and substituted or unsubstituted C₁₋₂₀-alkyl;
each m and n is independently 1 or 2; and
R^{a} are each independently selected from hydrogen and substituted or unsubstituted C₁₋₂₀-alkyl, substituted or unsubstituted C₁₋₂₀-heteroalkyl, and substituted or unsubstituted C₃₋₂₀-cycloalkyl.

Even more preferably, the transition metal catalyst comprises a tridentate ligand having a Formula (I), wherein:
X and Y are each —SR^{a};
R¹, R^{x}, R², R^{y}, R^{3a}, R^{3b}, R^{4a}, R^{4b} and R⁵ are each hydrogen;
m and n are each 1; and
R^{a} are each independently substituted or unsubstituted C₁₋₂₀-alkyl, preferably C₁₋₁₀ alkyl.

Even more preferably, the transition metal catalyst comprises a tridentate ligand having a Formula (I), wherein:
X and Y are each -SEt;
R¹, R^{x}, R², R^{y}, R^{3a}, R^{3b}, R^{4a}, R^{4b} and R⁵ are each hydrogen; and
m and n are each 1.

In alternative preferred processes of the present invention, the transition metal catalyst comprises a tridentate ligand having a Formula (I), wherein:
X is a heteroatom and when taken together with R¹ it forms an optionally substituted heteroaromatic ring when R^{x} is absent;
Y is —PR^{a}R^{b};
R² and R^{y} are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl;
R^{3a}, R^{3b}, R^{4a}, R^{4b} and R⁵ are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl; each m and n is independently 1 or 2; and
R^{a} and R^{b} are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl; or R^{a} and R^{b} together with the heteroatom to which they are attached form a heterocycle.

Preferably, the transition metal catalyst comprises a tridentate ligand having a Formula (I), wherein:
X is a nitrogen atom and when taken together with R¹ it forms an optionally substituted heteroaromatic ring when R^{x} is absent, wherein the heteroaromatic ring is a nitrogen-containing heteroaromatic ring;
Y is —PR^{a}R^{b};
R² and R^{y} are each independently is selected from hydrogen, substituted or unsubstituted C₁₋₂₀-alkyl, substituted or unsubstituted C₁₋₂₀-heteroalkyl, and substituted or unsubstituted C₃₋₂₀-cycloalkyl;
R^{3a}, R^{3b}, R^{4a}, R^{4b} and R⁵ are each independently selected from hydrogen, substituted or unsubstituted C₁₋₂₀-alkyl, substituted or unsubstituted C₁₋₂₀-heteroalkyl, substituted or unsubstituted C₃₋₂₀-cycloalkyl;
each m and n is independently 1 or 2; and
R^{a} and R^{b} are each independently selected from substituted or unsubstituted C₁₋₂₀-alkyl, substituted or unsubstituted C₁₋₂₀-heteroalkyl, substituted or unsubstituted C₃₋₂₀-cycloalkyl, substituted or unsubstituted C₅₋₂₀-aryl, and substituted or unsubstituted C₄₋₂₀-heteroaryl.

More preferably, the transition metal catalyst comprises a tridentate ligand having a Formula (I), wherein:
X is a nitrogen atom and when taken together with R¹ it forms an optionally substituted heteroaromatic ring when R^{x} is absent, wherein the heteroaromatic ring is a nitrogen-containing heteroaromatic ring selected from pyridinyl, pyrrolyl, imidazolyl, pyrazolyl, thiazolyl, isothiazolyl, oxazolyl, isoxazolyl, triazolyl, tetrazolyl, thiadiazolyl, oxadiazolyl, pyrimidyl, benzoxazolyl, benzthiazolyl, benzimidazolyl, indolyl, and quinolinyl;
Y is —PR^{a}R^{b};
R², R^{y}, R^{3a}, R^{3b}, R^{4a}, R^{4b} and R⁵ are each hydrogen;
each m and n is 1; and
R^{a} and R^{b} are each independently selected from substituted or unsubstituted C₁₋₂₀-alkyl and substituted or unsubstituted C₆₋₂₀-aryl.

Even more preferably, the transition metal catalyst comprises a tridentate ligand having a Formula (I), wherein:
X is a nitrogen atom and when taken together with R¹ it forms an optionally substituted heteroaromatic ring when R^{x} is absent, wherein the heteroaromatic ring is a nitrogen-containing heteroaromatic ring selected from pyridinyl, pyrrolyl, imidazolyl, pyrazolyl, triazolyl, tetrazolyl, and pyrimidyl;
Y is —PR^{a}R^{b};
R², R^{y}, R^{3a}, R^{3b}, R^{4a}, R^{4b} and R⁵ are each hydrogen;
each m and n is 1; and
R^{a} and R^{b} are each independently selected from methyl, ethyl, n-propyl, iso-propyl, n-butyl, sec-butyl, tert-butyl, pentyl, hexyl, phenyl, tolyl, xylyl, and methoxyphenyl.

Even more preferably, the transition metal catalyst comprises a tridentate ligand having a Formula (I), wherein:
X is a nitrogen atom and when taken together with R¹ it forms an optionally substituted pyridinyl ring when R^{x} is absent;
Y is —PR^{a}R^{b};
R², R^{y}, R^{3a}, R^{3b}, R^{4a}, R^{4b} and R⁵ are each hydrogen;
each m and n is 1; and
R^{a} and R^{b} are each independently selected from methyl, ethyl, iso-propyl, tert-butyl, phenyl, tolyl, xylyl, and methoxyphenyl.

Even more preferably, the transition metal catalyst comprises a tridentate ligand having a Formula (I), wherein:
X is a nitrogen atom and when taken together with R¹ it forms an optionally substituted pyridinyl ring when R^{x} is absent;
Y is —PR^{a}R^{b};
R², R^{y}, R^{3a}, R^{3b}, R^{4a}, R^{4b} and R⁵ are each hydrogen;
each m and n is 1; and
R^{a} and R^{b} are each phenyl.

In preferred processes of the present invention, the transition metal catalyst has a Formula (II) or Formula (III)

[M(L¹)(L²)_{d}] (II)

[M(L¹)(L²)_{d}]W (III)

wherein:
M is a transition metal;
L¹ is a tridentate ligand as hereinbefore defined;
L² are ligands which may be the same or different;
d is 1, 2 or 3; and
W is a non-coordinated anionic ligand.

In preferred processes of the present invention, M is a Group 6, Group 7, Group 8, or Group 9 transition metal. More preferably, M is a Group 7, Group 8, or Group 9 transition metal. Even more preferably, M is a Group 8 transition metal.

In preferred processes of the present invention, M is a transition metal selected from Mo, Mn, Fe, Ru, Co and Os. More preferably, M is a transition metal selected from Ru and Os. Most preferably, M is Ru.

In preferred processes of the present invention, d is 3.

As will be understood by a skilled person, each L² may be a monodentate ligand or a multidentate ligand, provided the combination of L² ligands is allowed by the rules of valency. In preferred processes of the present invention, each L² is a monodentate ligand. Preferably, each L² is independently a neutral monodentate ligand or an anionic monodentate ligand. In preferred processes of the present invention, each L² is independently selected from —H, —CO, —CN, —P(R′)₃, —As(R′)₃, —CR′, —OR′, —O(C═O)R′, —NR′₂, halogen (e.g. —Cl, —Br, —I), and solvent, wherein each R′ is independently selected from substituted or unsubstituted alkyl, substituted or unsubstituted alkenyl, substituted or unsubstituted alkynyl, substituted or unsubstituted heteroalkyl, substituted or unsubstituted alkoxy, substituted or unsubstituted cycloalkyl, substituted or unsubstituted heterocycloalkyl, substituted or unsubstituted aryl, and substituted or unsubstituted heteroaryl. Preferably, each L² is independently selected from —H, —CO, —P(R′)₃, and halogen. More preferably, each L² is independently selected from —CO, —PPh₃, and —Cl. When L² is solvent, the solvent is preferably selected from THF, Me-THF, MeCN, H₂O and an alcohol (e.g. methanol, ethanol, iso-propanol etc.).

In the transition metal catalysts of formula (III), W is a non-coordinated anionic ligand. By “non-coordinated anion ligand”, we mean the anionic ligand is forced to the outer sphere of the metal centre. The anionic ligand, therefore, is dissociated from the metal centre. This is in contrast to neutral complexes in which the anionic ligand is bound to the metal within the coordination sphere. The anionic ligand can be generally identified as non-coordinating by analysing the X-ray crystal structure of the cationic complex. Preferably, W is selected from the group consisting of triflate (i.e. TfO⁻ or CF₃SO₃⁻), tetrafluoroborate (i.e. —BF₄), hexafluoroantimonate (i.e. —SbF₆), hexafluorophosphate (PF₆⁻), [B[3,5-(CF₃)₂C₆H₃]₄]⁻ ([BAr^{F}₄]⁺), halide (e.g. Cl⁻, Br⁻, I⁻) and mesylate (MsO⁻ or MeSO₃⁻).

Preferably, the transition metal catalyst is a transition metal catalyst of Formula (II).

Alternatively, the transition metal catalyst is a transition metal catalyst of Formula (III).

In preferred processes of the present invention, the transition metal catalyst is

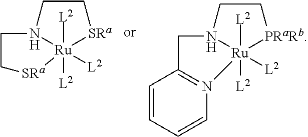

In preferred processes of the present invention, the transition metal catalyst is Ru-SNS or Ru-PNN.

In preferred processes of the present invention, the transition metal catalyst is

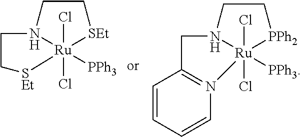

In preferred processes of the present invention, the transition metal catalyst employed in the processes of the present invention comprises a bidentate ligand.

In preferred processes of the present invention, the transition metal catalyst comprises a bidentate ligand having a Formula (IV)

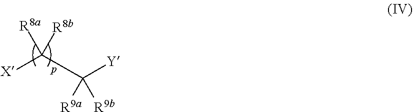

wherein:
X′ is —NHR^{ax};
Y′ is selected from —SR^{ax}, —OR^{ax}, —CR^{ax}, —NR^{ax}R^{bx}, —PR^{ax}R^{bx}, —P(═O)R^{ax}R^{bx}, —OPR^{ax}R^{bx}, and —NHPR^{ax}R^{bx};
R^{8a}, R^{8b}, R^{9a} and R^{9b} are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₅₋₂₀-aryl, and substituted or unsubstituted C₄₋₂₀-heteroaryl;
p is 1 or 2; and
R^{ax} and R^{bx}, if present, are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl; or when X′ and/or Y′ is —NR^{ax}R^{bx}, —PR^{ax}R^{bx}, —OPR^{ax}R^{bx}, or —NHPR^{ax}R^{bx}, R^{ax} and R^{bx} together with the heteroatom to which they are attached form a heterocycle.

In preferred processes of the present invention, the transition metal catalyst has a Formula (V) or Formula (VI)

[M(L¹)ₑ(L²)_{f}] (V)

[M(L¹)ₑ(L²)_{f}]W (VI)
wherein:
M is a transition metal;
L¹ are bidentate ligands as hereinbefore defined which may be the same or different;
L², if present, are ligands which may be the same or different;
e is 1 or 2 such that when e is 1, f is 2, 3 or 4, and when e is 2, f is 0, 1 or 2; and
W is a non-coordinated anionic ligand.

M, L² and W are as generally described above.

In preferred processes of the present invention, the transition metal catalyst employed in the processes of the present invention comprises a tetradentate ligand.

In preferred processes of the present invention, the transition metal catalyst comprises a tetradentate ligand having a Formula (VII)

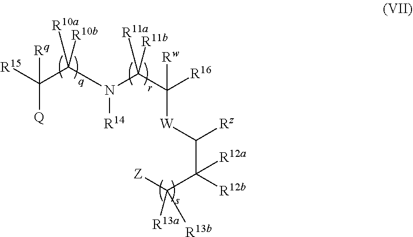

wherein:
Q is selected from —SR^{ay}, —OR^{y}, —CR^{ay}, —NR^{ay}R^{by}, —PR^{ay}R^{by}, —P(═O)R^{ay}R^{by}, —OPR^{ay}R^{by}, and —NHPR^{ay}R^{by};
R¹⁵ and R^{q} are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl;
or Q is a heteroatom and when taken together with R¹⁵ it forms an optionally substituted heterocycle when R^{q} is absent;
W is selected from S, O, NR^{a}, and PR^{a};
R¹⁶, R^{w} and R^{Z} are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl;
or R¹⁶ when taken together with R^{Z} forms an optionally substituted heterocycle when R^{w} is absent;
Z is selected from —SR^{ay}, —OR^{ay}, —CR^{ay}, —NR^{ay}R^{by}, —PR^{ay}R^{by}, —P(═O)R^{ay}R^{by}, —OPR^{a}R^{by}, and —NHPR^{ay}R^{by};
R¹⁰a, R^{10b}, R^{11a} and R^{11b} are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl; or
R^{10a} and one of R^{11a} and R^{11b} or R^{10b} and one of R^{11a} and R^{11b} together with the atoms to which they are bound, form a heterocycle;
R^{12a}, R^{12b}, R^{13a}, R^{13b} and R¹⁴ are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl; each q and r is independently 1 or 2;
s is 0, 1 or 2; and
R^{ay} and R^{by}, if present, are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl; or when Q and/or Z is —NR^{ay}R^{by}, —PR^{ay}R^{by}, —OPR^{ay}R^{by}, or —NHPR^{a}R^{by}, R^{ay} and R^{by} together with the heteroatom to which they are attached form a heterocycle.

In preferred processes of the present invention, the transition metal catalyst has a Formula (VIII) or Formula (IX)

[M(L¹)(L²)_{g}] (VIII)

[M(L¹)(L²)_{g}] (IX)

wherein:
M is a transition metal;
L¹ is a tetradentate ligand as hereinbefore defined;
L², if present, are ligands which may be the same or different;
g is 0, 1 or 2; and
W is a non-coordinated anionic ligand.

M, L² and W are as generally described above.

In preferred processes of the present invention, the transition metal catalyst is removed from the reaction mixture by a precipitation step using a co-solvent.

In alternative preferred processes of the present invention, the transition metal catalyst is removed from the reaction mixture by distillation of the product.

In alternative preferred processes of the present invention, the transition metal catalyst is removed from the reaction mixture by crystallisation of the product.

In alternative preferred processes of the present invention, the transition metal catalyst is removed from the reaction mixture using a metal scavenger.

In preferred processes of the present invention, the ester-containing substrate contains at least one ester moiety.

In preferred processes of the present invention, the ester-containing substrate contains one ester moiety. Preferably, the ester-containing substrate is of Formula (X)

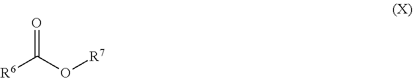

wherein:
R⁶ and R⁷ are independently organic groups having 1-70 carbon atoms; or
R⁶/R⁷ forms a ring structure with the atoms to which they are attached.

In preferred processes of the present invention, R⁶ and R⁷ are independently organic groups having 1-70 carbon atoms.

In preferred processes of the present invention, R⁶ is selected from substituted or unsubstituted C₁₋₇₀-alkyl, substituted or unsubstituted C₂₋₇₀-alkenyl, substituted or unsubstituted C₂₋₇₀-alkynyl, substituted or unsubstituted C₁₋₇₀-heteroalkyl, substituted or unsubstituted C₃₋₇₀-cycloalkyl, substituted or unsubstituted C₃₋₇₀-cycloalkenyl, substituted or unsubstituted C₂₋₇₀-heterocycloalkyl, substituted or unsubstituted C₆₋₇₀-aryl, and substituted or unsubstituted C₄₋₇₀-heteroaryl, preferably substituted or unsubstituted C₁₋₅₀-alkyl, substituted or unsubstituted C₂₋₅₀-alkenyl, substituted or unsubstituted C₂₋₅₀-alkynyl, substituted or unsubstituted C₁₋₅₀-heteroalkyl, substituted or unsubstituted C₃₋₅₀-cycloalkyl, substituted or unsubstituted C₃₋₅₀-cycloalkenyl, substituted or unsubstituted C₂₋₅₀-heterocycloalkyl, substituted or unsubstituted C₆₋₅₀-aryl, and substituted or unsubstituted C₄₋₅₀-heteroaryl, more preferably substituted or unsubstituted C₁₋₃₀-alkyl, substituted or unsubstituted C₂₋₃₀-alkenyl, substituted or unsubstituted C₂₋₃₀-alkynyl, substituted or unsubstituted C₁₋₃₀-heteroalkyl, substituted or unsubstituted C₃₋₃₀-cycloalkyl, substituted or unsubstituted C₃₋₃₀-cycloalkenyl, substituted or unsubstituted C₂₋₃₀-heterocycloalkyl, substituted or unsubstituted C₆₋₃₀-aryl, and substituted or unsubstituted C₄₋₃₀-heteroaryl, even more preferably substituted or unsubstituted C₁₋₂₀-alkyl (e.g. C₃₋₂₀-alkyl), substituted or unsubstituted C₂₋₂₀-alkenyl (e.g. C₈₋₂₀-alkenyl), substituted or unsubstituted C₂₋₂₀-alkynyl (e.g. C₃₋₂₀-alkynyl), substituted or unsubstituted C₁₋₂₀-heteroalkyl (e.g. C₃₋₂₀-heteroalkyl), substituted or unsubstituted C₃₋₂₀-cycloalkyl (e.g. C₃₋₂₀-cycloalkyl), substituted or unsubstituted C₃₋₂₀-cycloalkenyl (e.g. C₃₋₂₀-cycloalkenyl), substituted or unsubstituted C₂₋₂₀-heterocycloalkyl (e.g. C₃₋₂₀-heterocycloalkyl), substituted or unsubstituted C₆₋₂₀-aryl (e.g. C₃₋₂₀-aryl), and substituted or unsubstituted C₄₋₂₀-heteroaryl (e.g. C₃₋₂₀-heteroaryl). Preferably, R⁶ is selected from substituted or unsubstituted C₁₋₇₀-alkyl, substituted or unsubstituted C₂₋₇₀-alkenyl, substituted or unsubstituted C₁₋₇₀-heteroalkyl, substituted or unsubstituted C₆₋₇₀-aryl, and substituted or unsubstituted C₄₋₇₀-heteroaryl, more preferably substituted or unsubstituted C₁₋₅₀-alkyl, substituted or unsubstituted C₂₋₅₀-alkenyl, substituted or unsubstituted C₁₋₅₀-heteroalkyl, substituted or unsubstituted C₆₋₅₀-aryl, and substituted or unsubstituted C₄₋₅₀-heteroaryl, even more preferably substituted or unsubstituted C₁₋₃₀-alkyl, substituted or unsubstituted C₂₋₃₀-alkenyl, substituted or unsubstituted C₁₋₃₀-heteroalkyl, substituted or unsubstituted C₆₋₃₀-aryl, and substituted or unsubstituted C₄₋₃₀-heteroaryl, even more preferably substituted or unsubstituted C₁₋₂₀-alkyl (e.g. C₃₋₂₀-alkyl), substituted or unsubstituted C₂₋₂₀-alkenyl (e.g. C₈₋₂₀-alkenyl), substituted or unsubstituted C₁₋₂₀-heteroalkyl (e.g. C₃₋₂₀-heteroalkyl), substituted or unsubstituted C₆₋₂₀-aryl (e.g. C₈₋₂₀-aryl), and substituted or unsubstituted C₄₋₂₀-heteroaryl (e.g. C₃₋₂₀-heteroaryl). More preferably, R⁶ is selected from substituted or unsubstituted C₁₋₇₀-alkyl, substituted or unsubstituted C₂₋₇₀-alkenyl, and substituted or unsubstituted C₆₋₇₀-aryl, more preferably substituted or unsubstituted C₁₋₅₀-alkyl, substituted or unsubstituted C₂₋₅₀-alkenyl, and substituted or unsubstituted C₆₋₅₀-aryl, even more preferably substituted or unsubstituted C₁₋₃₀-alkyl, substituted or unsubstituted C₂₋₃₀-alkenyl, and substituted or unsubstituted C₆₋₃₀-aryl, even more preferably substituted or unsubstituted C₁₋₂₀-alkyl (e.g. C₃₋₂₀-alkyl), substituted or unsubstituted C₂₋₂₀-alkenyl (e.g. C₃₋₂₀-alkenyl), and substituted or unsubstituted C₆₋₂₀-aryl (e.g. C₃₋₂₀-aryl). More preferably, R⁶ is selected from substituted or unsubstituted C₁₋₇₀-alkyl and substituted or unsubstituted C₆₋₇₀-aryl, more preferably substituted or unsubstituted C₁₋₅₀-alkyl and substituted or unsubstituted C₆₋₅₀-aryl, even more preferably substituted or unsubstituted C₁₋₃₀-alkyl and substituted or unsubstituted C₆₋₃₀-aryl, even more preferably substituted or unsubstituted C₁₋₂₀-alkyl (e.g. C₃₋₂₀-alkyl) and substituted or unsubstituted C₆₋₂₀-aryl (e.g. C₃₋₂₀-aryl). Even more preferably, R⁶ is selected from methyl, ethyl, n-propyl, iso-propyl, n-butyl, sec-butyl, tert-butyl, pentyl, hexyl, heptyl, octyl, phenyl, tolyl, xylyl, and methoxyphenyl.

In alternative preferred processes of the present invention, R⁶ is an aliphatic group containing at least 9 carbon atoms. Preferably, R⁶ is selected from substituted or unsubstituted C₉₋₇₀-alkyl, substituted or unsubstituted C₉₋₇₀-alkenyl, substituted or unsubstituted C₉₋₇₀-cycloalkyl, and substituted or unsubstituted C₉₋₇₀-cycloalkenyl, more preferably substituted or unsubstituted C₉₋₅₀-alkyl, substituted or unsubstituted C₉₋₅₀-alkenyl, substituted or unsubstituted C₉₋₅₀-cycloalkyl, and substituted or unsubstituted C₉₋₅₀-cycloalkenyl, more preferably substituted or unsubstituted C₉₋₃₀-alkyl, substituted or unsubstituted C₉₋₃₀-alkenyl, substituted or unsubstituted C₉₋₃₀-cycloalkyl, and substituted or unsubstituted C₉₋₃₀-cycloalkenyl, even more preferably substituted or unsubstituted C₉₋₂₀-alkyl, substituted or unsubstituted C₉₋₂₀-alkenyl, substituted or unsubstituted C₉₋₂₀-cycloalkyl, and substituted or unsubstituted C₉₋₂₀-cycloalkenyl.

In preferred processes of the present invention, R⁷ is selected from substituted or unsubstituted C₁₋₇₀-alkyl or substituted or unsubstituted C₆₋₇₀-aryl, preferably substituted or unsubstituted C₁₋₅₀-alkyl or substituted or unsubstituted C₆₋₅₀-aryl, more preferably substituted or unsubstituted C₁₋₃₀-alkyl or substituted or unsubstituted C₆₋₃₀-aryl, even more preferably substituted or unsubstituted C₁₋₂₀-alkyl (e.g. C₃₋₂₀-alkyl) or substituted or unsubstituted C₆₋₂₀-aryl (e.g. C₃₋₂₀-aryl). Preferably, R⁷ is selected from substituted or unsubstituted C₁₋₇₀-alkyl, more preferably substituted or unsubstituted C₁₋₅₀-alkyl, even more preferably substituted or unsubstituted C₁₋₃₀-alkyl, even more preferably substituted or unsubstituted C₁₋₂₀-alkyl (e.g. C₈₋₂₀-alkyl). More preferably, R⁷ is selected from methyl, ethyl, n-propyl, iso-propyl, n-butyl, sec-butyl, tert-butyl, pentyl, hexyl, heptyl, and octyl.

In preferred processes of the present invention, the ester-containing substrate is a methyl ester (e.g. methyl acetate).

In preferred processes of the present invention, the ester-containing substrate is an ethyl ester (e.g. ethyl acetate).

In preferred processes of the present invention, the ester-containing substrate is a wax ester.

In preferred processes of the present invention, R⁶/R⁷ forms a ring structure with the atoms to which they are attached (i.e. the ring structure is a lactone). Preferably, the ring structure is a 4 to 7 membered ring system. More preferably, the ring structure is a 5 to 6 membered ring system.

In preferred processes of the present invention, the ester-containing substrate contains more than one ester moiety, preferably two or three ester moieties.

In preferred processes of the present invention, the ester-containing substrate is selected from

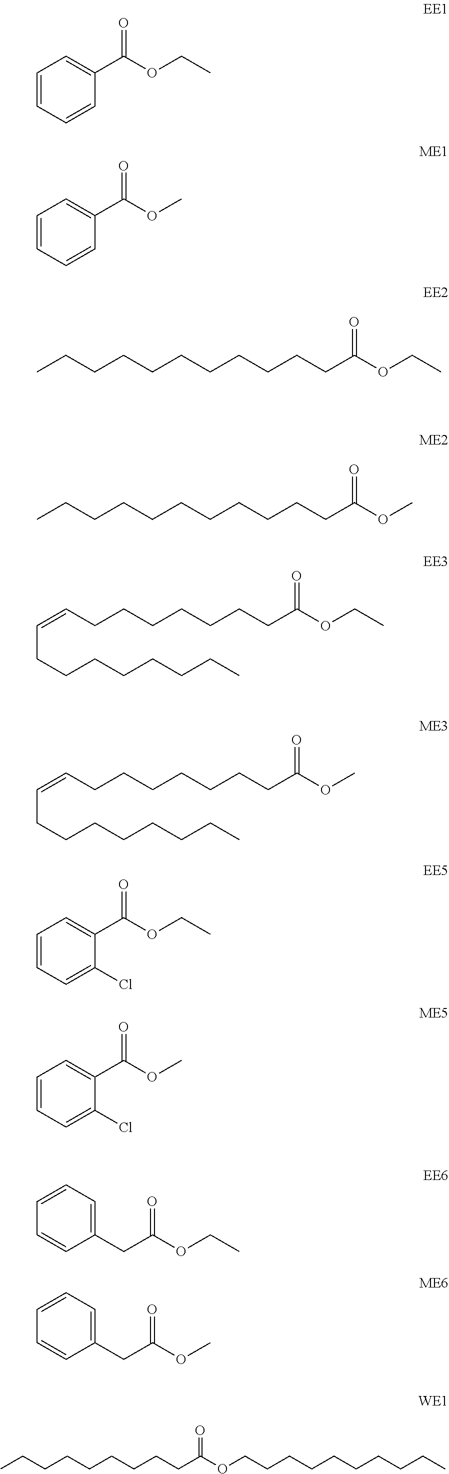

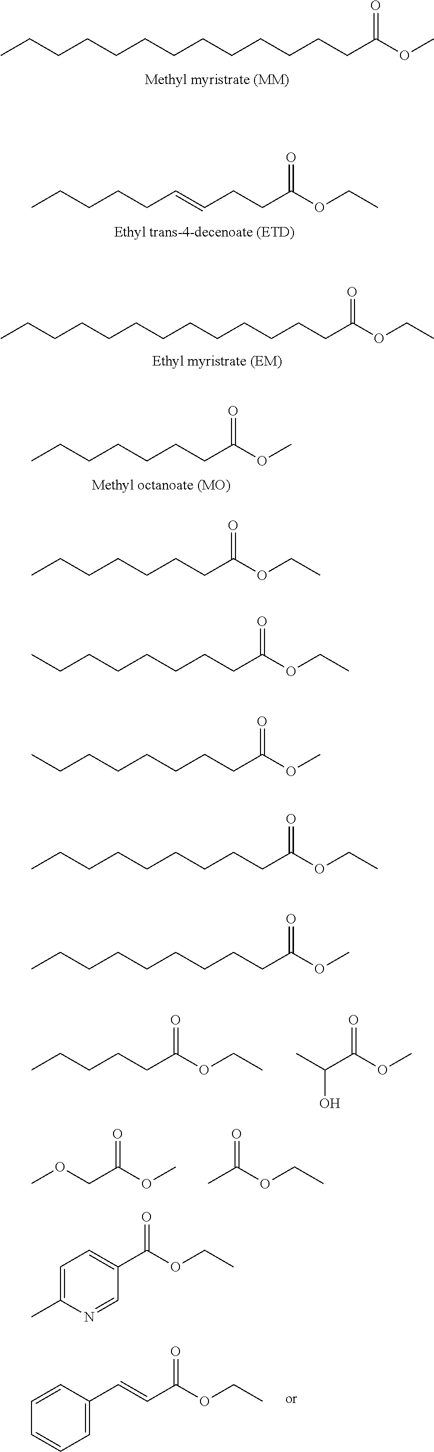

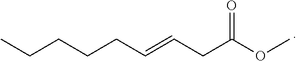

When the ester-containing substrate is a monoester, the products of the process of the present invention are alcohols. When the ester-containing substrate is a lactone, the product of the process of the present invention is a diol. When the ester-containing substrate contains multiple ester moieties, the products of the process of the present invention are a polyol and alcohols. In preferred processes of the present invention, the process does not produce a hemiacetal by-product.

In the instance the ester-containing substrate contains an alkenyl and/or an alkynyl moiety, the process of the present invention preferably selectively hydrogenates the ester moiety over the unsaturated carbon-carbon bond of the alkene and/or alkyne. Alternatively, in the instance the ester-containing substrate contains an alkenyl and/or an alkynyl moiety, the process of the present invention preferably hydrogenates both the ester moiety and the unsaturated carbon-carbon bond of the alkene and/or alkyne.

In the instance the ester-containing substrate contains a ketone and/or an aldehyde moiety, the process of the present invention preferably hydrogenates both the ester moiety and the carbon-oxygen double bond of the ketone and/or aldehyde.

In the instance the ester-containing substrate contains an alkenyl and/or an alkynyl moiety, and a ketone and/or an aldehyde moiety, the process of the present invention preferably selectively hydrogenates both the ester moiety and the carbon-oxygen double bond of the ketone and/or aldehyde over the unsaturated carbon-carbon bond of the alkene and/or alkyne. Alternatively, in the instance the ester-containing substrate contains an alkenyl and/or an alkynyl moiety, and a ketone and/or an aldehyde moiety, the process of the present invention preferably hydrogenates the ester moiety, the unsaturated carbon-carbon bond of the alkene and/or alkyne, and the carbon-oxygen double bond of the ketone and/or aldehyde.

As will be understood by a skilled person, an alkoxide base is added and remains present during the hydrogenation process of the present invention. The alkoxide base can be recovered from the reaction mixture and recycled or reused in a subsequent hydrogenation process of the present invention. For example, if the ester-containing substrate is ethyl acetate and the base is sodium ethoxide, then the hydrogenation reaction will generate ethanol and return sodium ethoxide as products and the recovered sodium ethoxide can then be recycled or reused. For an alternative example, if the ester-containing substrate is ethyl decanoate and the base is sodium ethoxide, then the hydrogenation reaction will generate ethanol and decanol. In this case, the recovered and reusable alkoxide base can be sodium ethoxide and/or sodium decanoate.

In preferred processes of the present invention, the process is a batch process. Preferably, the process is a batch process wherein any excess base is recycled or reused. The base may be recycled or reused once, twice, three times, or more.

In preferred processes of the present invention, the process is a flow process. Preferably, the process is a flow process wherein any excess base is recycled or reused. The base may be recycled or reused once, twice, three times, or more.

The invention will now be further described by way of the following non-limiting examples.

### Examples

### Materials

Ru-SNS and Ru-PNN are commercially available from Johnson Matthey.

Ru-SNN was made according to the procedure outlined in Puylaert et al., Chem. Eur. J. 2017, 23, 8473-8481.

NaOEt and the ester-containing substrates EE1, EE2, EE3, EE5, EE6, ME1, ME2, ME6, WE1, MM, EM, ETD, ED, MO, and ethyl acetate are all commercially available, e.g. from Sigma Aldrich, Fisher Scientific, Alfa Aesar, Acros Organics etc.

### Measurement Methods

Gas chromatography (GC) measurements were conducted using a Varian 3900 or 3800 gas chromatograph system. Unless otherwise indicated, reaction conversions were determined by GC analysis.

Nuclear magnetic resonance (NMR) measurements were conducted using a Bruker Avance Ill 400 (400 MHz) spectrometer.

### General Procedure for Ester Hydrogenation

To an 8 mL vial, a catalyst Ru-SNS, Ru-PNN, Ru-SNN was added, followed by solid base, NaOEt, and then 10-20 mmol of ester-containing substrate. The vial was then added to a Biotage Endeavor screening system, before the stirring head was sealed and the reaction mixture purged with nitrogen. A purge sequence involved pressurizing to approximately 45 psi nitrogen and then releasing the pressure (repeated 5 times). The reactor was then pressurised with hydrogen, then heated and pressurized to a set pressure of hydrogen. Once the reaction time was complete, typically 16 hrs, the reaction was allowed to cool to room temperature. Nitrogen purge cycles (5 repeats) were then carried out to remove hydrogen. The reaction mixture was then analysed via GC and NMR.

### Example 1: Ester Hydrogenation

The General Procedure described above was carried out on the following ester-containing substrates:

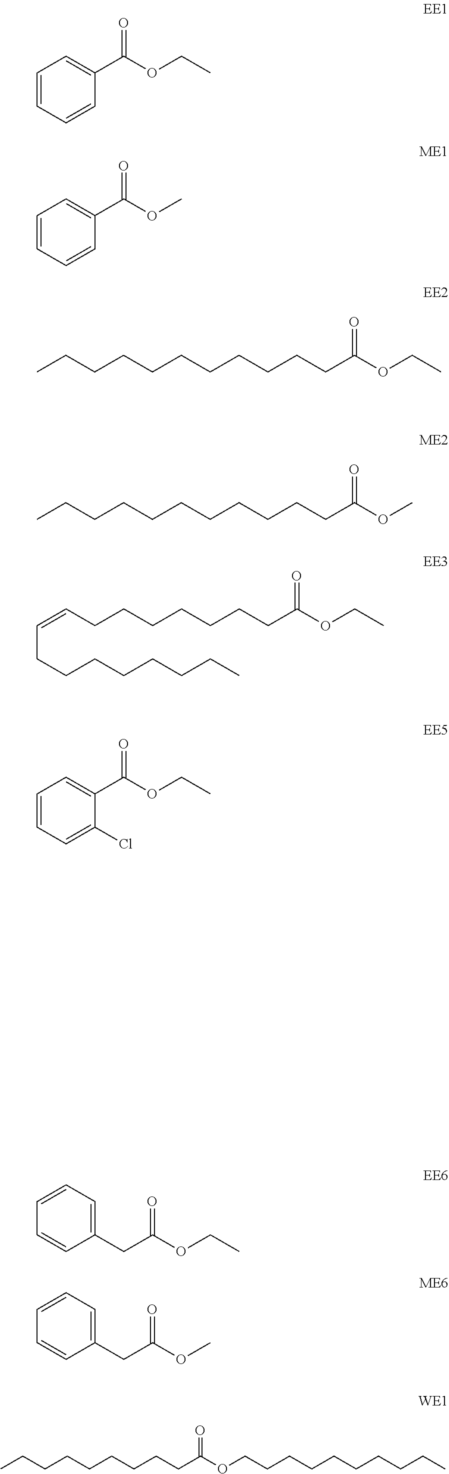

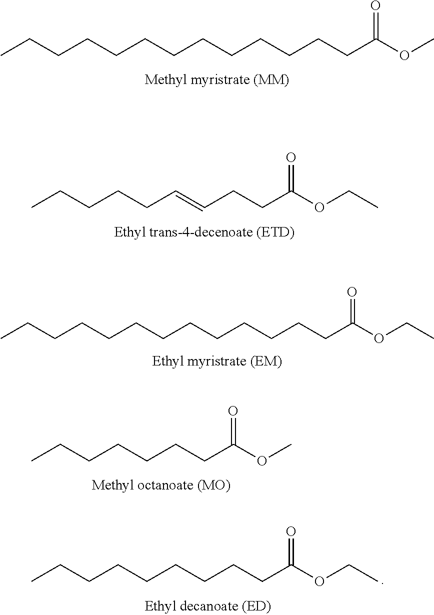

The conditions used in each reaction are listed in Tables 1a and 1b below. The results of each reaction, in terms of conversion and TON, are also listed in Tables 1a and 1b. In the instances where the substrate was EE3 and ETD, the carbon-carbon double bond was not reduced under the reaction conditions (i.e. the process was selective for reduction of the ester moiety).

The results in Tables 1a and 1b show that the process of the present invention can achieve high conversion and industrially useful TON for the ester hydrogenation of a wide range of ester-containing substrates, including methyl esters, ethyl esters and even wax esters, at extremely low catalyst loadings (e.g. 400,000/1 S/C or lower). The results also show that a range of different transition metal catalysts containing tridentate ligands can be used to perform the hydrogenation reaction.

Entry 1 of Table 1a further demonstrates that even at hydrogen pressures of as low as 5 bar, efficient turnover (TON=48650) and conversion (97.3%) can still be achieved at very low catalyst loadings (50,000/1 S/C, EE1/Ru-PNN).

Entry 3 of Table 1a further shows that an economical ester hydrogenation process can be achieved at an extremely low catalyst loading (400,000/1 S/C, EE1/Ru-SNS), as a very high TON of 324000 was achieved. The reaction proceeded with an 81.0% conversion, which is thought to be due to the trade-off between catalyst activity and conversion that occurs at very low catalyst loadings. With such a high TON value, the partial conversion is not an issue as the starting materials and products are easily separable. The recovered starting material can then be re-subjected to the ester hydrogenation process of the present invention.

Entries 5 and 6 of Table 1b further show that the use of small amounts of solvent (e.g. EtOH) is not determinantal to conversion or TON. To the contrary, the inventors have found that the use of solvent in this way can help improve reaction performance for certain types of substrate.

### Example 2a: Variation of Amount of Base

The General Procedure described above was carried out using ethyl dodecanoate (EE2) as the ester-containing substrate and either Ru-SNS or Ru-PNN as catalyst (at a S/C loading of 100,000/1). Temperature, pressure, catalyst loading and substrate amount were kept constant, but the amount of solid NaOEt base was altered. The results are shown in Table 2a below.

The results in Table 2a clearly show that when the amount of solid NaOEt base in the reaction mixture is increased, the conversion for the ester hydrogenation reaction increases dramatically despite the S/C catalyst loading remaining extremely low (i.e. 100,000/1). This was observed for both sets of experiments, i.e. those involving the Ru-SNS catalyst and those involving the Ru-PNN catalyst. In each set of experiments, conversion increases as the base is increased from 10 mol % up to 40 mol %. Furthermore, when the amount of base is at least 30 mol %, conversions of over 96% can be achieved. Significantly lower conversions are observed at amounts of base less than 30 mol %, e.g. at 20 mol % base a conversion of 85.7% was observed for Ru-SNS and a conversion of 59.3% was observed for Ru-PNN, whilst at 10 mol % base a conversion 53.5% was observed for Ru-SNS and a conversion of 25.4% was observed for Ru-PNN.

### Example 2b: Variation of Amount of Base

The General Procedure described above was carried out using ethyl decanoate (ED) as the ester-containing substrate and Ru-PNN as catalyst (at a S/C loading of 100,000/1). Temperature, pressure, catalyst loading and substrate amount were kept constant, but the amount of solid NaOEt base was altered. In some experiments, the amount of base employed was greater than that employed in the experiments of Example 2a. The results are shown in Table 2b below.

The results in Table 2b show that when the amount of solid NaOEt base in the reaction mixture is increased, the conversion for the ester hydrogenation reaction increases despite the S/C catalyst loading remaining extremely low (i.e. 100,000/1). In these experiments, conversion increased as the base increased from 30 mol % up to 50 mol %.

### Example 2c: Variation of Amount of Base

The General Procedure described above was carried out using ethyl decanoate (ED) as the ester-containing substrate and either Ru-SNS or Ru-PNN as catalyst (at a S/C loading of 50,000/1). Temperature, pressure, catalyst loading and substrate amount were kept constant, but the amount of solid NaOEt base was altered. The results are shown in Table 2c below.

Compared to the experiments described in Examples 2a and 2b, the experiments of Example 2c were conducted at a lower hydrogen pressure of 12 bar. This means that the effect of the change in the amount of base is more pronounced as the reaction is limited by the lower hydrogen concentration. The results in Table 2c show that higher activity was observed in reactions using more base: for both Ru-SNS and Ru-PNN, conversion gradually increased as the amount of base used also increased. Turnover frequency (TOF) was found to follow a similar pattern to reaction conversion, with values for the Ru-PNN-catalysed reactions being higher than those catalysed by Ru-SNS suggesting Ru-PNN is a more active catalyst at lower hydrogen pressure. Example 2c therefore shows that using higher amounts of base can improve catalytic activity and conversion at low hydrogen pressure.

### Example 3a: Variation of Temperature

The General Procedure described above was carried out using ethyl benzoate (EE1) as the ester-containing substrate and Ru-SNS as catalyst (at a S/C loading of either 50,000/1 or 100,000/1). Pressure and the amount of base were kept constant, but the temperature at which the reaction was conducted was altered. The results are shown in Table 3 below.

As a first point, it is clear that a catalyst is required to bring about the hydrogenation of this ester-containing substrate as 0% conversion was observed when no catalyst was employed (entry 1).

Entries 2 and 3 are repeats of the same experiment wherein the following conditions were used: 50,000/1 catalyst loading, 30 bar hydrogen pressure, 50 mol % NaOEt base, 20 mmol substrate, and 40° C. temperature. High conversions were observed for the hydrogenation reaction (99.0% and 99.2%, respectively). When the experiment was repeated using a higher temperature of 65° C., conversion decreased significantly to 79.0% (entry 4).

Entries 5 and 6 are repeats of the same experiment wherein the following conditions were used: 100,000/1 catalyst loading, 30 bar hydrogen pressure, 50 mol % NaOEt base, 20 mmol substrate, and 40° C. temperature. High conversions were observed for the hydrogenation reaction (99.0% and 99.4%, respectively). When the experiment was repeated using a higher temperature of 65° C., conversion decreased significantly to 82.5% (entry 7).

The results in Table 3 show that the ester hydrogenation reaction achieves higher conversion at lower temperatures.

### Examples 3b and 3c: Variation of Temperature

Ethyl oleate EE3 (technical grade, 70% from Alfa Aesar) was first analysed by GC for purity. The results showed that the ethyl oleate used in Examples 3b and 3c had the following composition, indicating that some side products were to be expected in the subsequent hydrogenation reactions.

| | | Compositional |
|---|---|---|
| | Structure | analysis |
| Linoleic C18:2 | 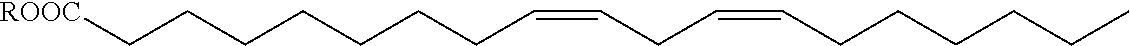 | 9.8% |
| Oleic C18:1 | 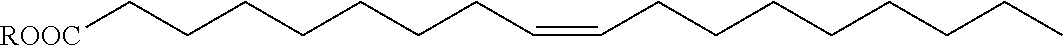 | 76.7% |
| Stearic C18:0 | 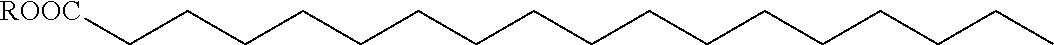 | 4.2% |
| Palmitic C16:0 | 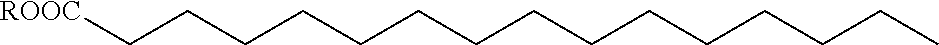 | 9.3% |

### Example 3b

The General Procedure described above was carried out using ethyl oleate (EE3) as the ester-containing substrate and Ru-SNS as catalyst. Pressure, substrate amount, and base amount were kept constant but the catalyst loading (S/C) and temperature at which the reaction was conducted was altered. The results are shown in Table 4 below.

At a catalyst loading of 25,000/1 (S/C), the C18:1 hydrogenation reaction conversion was similar when temperatures of 40° C. and 65° C. were employed and all other conditions were kept constant. When the temperature was raised to 80° C., a significant decrease in C18:1 conversion was observed (entries 1, 4 and 7).

At a catalyst loading of 50,000/1 (S/C), the C18:1 hydrogenation reaction conversion decreased from 100% to 93.2% when the temperature was raised from 40° C. to 65° C. and all other conditions were kept constant (entries 2 and 5).

At a catalyst loading of 100,000/1 (S/C), the C18:1 hydrogenation reaction conversion decreased dramatically from 99.1% to 9.7% when the temperature was raised from 40° C. and 65° C. and all other conditions were kept constant (entries 3 and 6).

The results in Table 4 show that the ester hydrogenation achieves higher C18:1 conversion at lower temperatures. Furthermore, the effect is more pronounced at lower catalyst loadings, with bigger decreases in C18:1 conversion being seen as temperature is increased.

### Example 3c

The General Procedure described above was carried out using ethyl oleate (EE3) as the ester-containing substrate and Ru-PNN as catalyst. Pressure, substrate amount, and base amount were kept constant but the catalyst loading (S/C) and temperature at which the reaction was conducted was altered. The results are shown in Table 5 below.

At a catalyst loading of 25,000/1 (S/C), the C18:1 hydrogenation reaction conversion was similar when temperatures of 40° C., 65° C. and 80° C. were employed and all other conditions were kept constant (entries 1, 4 and 7).

At a catalyst loading of 100,000/1 (S/C), the C18:1 hydrogenation reaction conversion decreased dramatically from 100% to 51.6% when the temperature was raised from 40° C. to 65° C. and all other conditions were kept constant (entries 3 and 6).

The results in Table 5 show that the ester hydrogenation achieves higher C18:1 conversion at lower temperatures. Again, the effect is more pronounced at lower catalyst loadings, with bigger decreases in C18:1 conversion being seen as temperature is increased.

The results of Examples 3a, 3b and 3c indicate that the ester hydrogenation reaction is sensitive to temperature and performs best in terms of reaction conversion at lower temperatures (e.g. −40° C.). This has been demonstrated across a range of ester-containing substrates and catalysts. A lower reaction temperature is particularly beneficial when very low catalyst loadings are used.

### Example 3d: Variation of Temperature

Methyl oleate (technical grade, C18′ 70-85% from ThermoFisher Scientific) was first analysed by GC for purity. The results showed that the methyl oleate used in Example 3d had the following composition, indicating that some side products were to be expected in the subsequent hydrogenation reactions.

| | | Compositional |
|---|---|---|
| | Structure | analysis |
| Linoleic C18:2 | 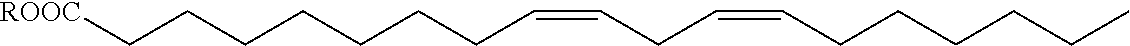 | 10.8% |
| Oleic C18:1 | 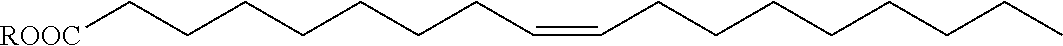 | 81.1% |
| Stearic C18:0 | 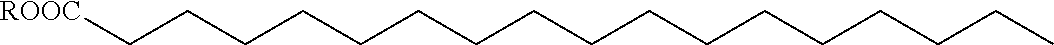 | 3.3% |
| Palmitic C16:0 | 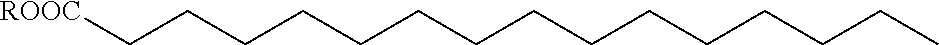 | 4.8% |

The General Procedure described above was carried out using methyl oleate as the ester-containing substrate and Ru-SNS as catalyst. Pressure, substrate amount, and base amount were kept constant but the catalyst loading (S/C) and temperature at which the reaction was conducted was altered. The results are shown in Table 6 below.

At a catalyst loading of 25,000/1 (S/C), the C18:1 hydrogenation reaction conversion was similar when temperatures of 40° C., 50° C. and 60° C. were employed and all other conditions were kept constant. When the temperature was raised to 70° C., a significant decrease in C18:1 conversion was observed.

At a catalyst loading of 50,000/1 (S/C), the C18:1 hydrogenation reaction conversion consistently decreased as the temperature was increased from 40° C. to 70° C. (in increments of 10° C.) and all other conditions were kept constant.

The results in Table 6 show that the ester hydrogenation achieves higher C18:1 conversion at lower temperatures. Again, the effect is more pronounced at lower catalyst loadings, with bigger decreases in C18:1 conversion being seen as temperature is increased.

### Example 4: Variation of Pressure

The ethyl oleate (EE3) sample used in Examples 3b and 3c was also employed in Example 4.

The General Procedure described above was carried out using ethyl oleate (EE3) as the ester-containing substrate and Ru-SNS or Ru-PNN as catalyst. In this Example, the ester hydrogenation reaction was conducted at a lower hydrogen pressure of 10 bar. The results are shown in Table 7 below.

Comparison of entry 1 of Table 7 with entry 2 of Table 4 shows comparable results. Thus, when all other variables remain constant, the ester hydrogenation reaction conducted using Ru-SNS achieves a high C18:1 conversion even at a much lower hydrogen pressure of 10 bar (compared to 30 bar in entry 2 of Table 4).

Comparison of entry 2 of Table 7 with entry 2 of Table 5 also shows comparable results. Thus, when all other variables remain constant, the ester hydrogenation reaction conducted using Ru-PNN achieves a high C18:1 conversion even at a much lower hydrogen pressure of 10 bar (compared to 30 bar in entry 2 of Table 5).

Being able to operate the ester hydrogenation process of the present invention at lower pressures offers the additional advantages of reduced cost and improved safety. In addition, the need for specialist equipment that must withstand high pressures is avoided.

### Example 5: Recycling/Reuse of Base

A series of experiments was performed to determine if it is possible to recycle/reuse the base from a first run of the hydrogenation reaction in a subsequent second run.

First Run

The General Procedure described above was carried out using ethyl acetate as the ester-containing substrate and Ru-PNN as catalyst (at a S/C loading of 50,000/1). The reaction was repeated three times using the conditions shown in Table 8a below.

Second Run

The ethanol product of the hydrogenation reaction was removed under vacuum from each of the reaction mixtures of the first run. The base recovered from each reaction (in this case, NaOEt) was then transferred to three separate vials for a second run. The vials were then prepared as follows:

Test (i), using reaction mixture of experiment 1 of Table 8a—addition of fresh ethyl acetate substrate (2 mL, 20.47 mmol), addition of fresh Ru-PNN catalyst (0.309 mg, 4.09×10⁻³ mmol), and addition of fresh NaOEt base to top up to 50 mol %;
Test (ii), using reaction mixture of experiment 2 of Table 8a—addition of fresh ethyl acetate substrate (2 mL, 20.47 mmol) and addition of fresh Ru-PNN catalyst (0.309 mg, 4.09×10⁻³ mmol).

The vials were then added to a Biotage Endeavor screening system, and the hydrogenation reactions carried out according to the General Procedure described above. The results are shown in Table 8b below.

The results show that the reaction carried out using only recycled/reused base (test (ii)) performed equally well in terms of conversion compared to the reaction topped up with fresh base (test (i)), and a similar rate of reaction was observed for both. The results indicate that the base generated in a first run of the hydrogenation reaction can be effectively recycled/reused in a second run. It is also envisaged that further recycling/reuse of the generated base could be carried out, e.g. in a third run. This has the potential to make the overall process more cost effective, especially on an industrial scale. The results also show that it is possible to top up the recycled/reused base with fresh base in order to mitigate for mechanical losses, e.g. when operating the process on a small scale.

TABLE 1a

| | | | | | | H₂ | | |
|---|---|---|---|---|---|---|---|---|
| | | | Loading | Base | Temperature | pressure | Conversion | |
| Entry | Ester | Catalyst | (S/C) | (mol %) | (° C.) | (bar) | (%) | TON |
| 1 | EE1 | Ru-PNN | 50000 | 50 | 40 | 5 | 97.3 | 48650 |
| 2 | EE1 | Ru-PNN | 100000 | 50 | 40 | 30 | 96.7 | 96700 |
| 3 | EE1 | Ru-SNS | 400000 | 50 | 40 | 30 | 81.0 | 324000 |
| 4 | EE1 | Ru-SNS | 200000 | 50 | 40 | 30 | 98.1 | 196200 |
| 5 | ME1 | Ru-SNS | 200000 | 50 | 40 | 30 | 41.2 | 82400 |
| 6 | ME1 | Ru-SNS | 100000 | 50 | 40 | 30 | 87.1 | 87100 |
| 7 | ME1 | Ru-SNS | 50000 | 50 | 40 | 30 | 94.2 | 47100 |
| 8 | EE2 | Ru-PNN | 200000 | 50 | 40 | 30 | 78.1 | 156200 |
| 9 | EE2 | Ru-PNN | 100000 | 40 | 40 | 30 | 99.9 | 99900 |
| 10 | EE2 | Ru-SNS | 200000 | 30 | 40 | 30 | 11.5 | 23000 |
| 11 | EE2 | Ru-SNS | 100000 | 40 | 40 | 30 | 97.4 | 97400 |
| 12 | ME2 | Ru-SNS | 50000 | 40 | 40 | 30 | 10.6 | 5300 |
| 13 | ME2 | Ru-SNS | 25000 | 40 | 40 | 30 | 99.2 | 24800 |
| 14 | EE3 | Ru-PNN | 400000 | 50 | 40 | 30 | 32.2 | 128800 |
| 15 | EE3 | Ru-PNN | 200000 | 50 | 40 | 30 | 92.5 | 185000 |
| 16 | EE3 | Ru-PNN | 100000 | 50 | 40 | 30 | 97.7 | 97700 |
| 17 | EE3 | Ru-SNS | 200000 | 50 | 40 | 30 | 96.4 | 192800 |
| 18 | EE3 | Ru-SNN | 100000 | 50 | 40 | 30 | 92.9 | 92900 |
| 19 | EE3 | Ru-SNN | 50000 | 50 | 40 | 30 | 96.2 | 48100 |
| 20 | EE5 | Ru-PNN | 50000 | 40 | 40 | 30 | 77.5 | 38750 |
| 21 | EE5 | Ru-SNS | 40000 | 40 | 40 | 30 | 96.4 | 38560 |
| 22 | EE6 | Ru-PNN | 100000 | 50 | 65 | 30 | 25.2 | 25200 |
| 23 | EE6 | Ru-PNN | 25000 | 50 | 40 | 30 | 97.3 | 24325 |
| 24 | EE6 | Ru-SNS | 100000 | 50 | 40 | 30 | 19.7 | 19700 |
| 25 | EE6 | Ru-SNS | 50000 | 50 | 40 | 30 | 80.1 | 40050 |
| 26 | ME6 | Ru-SNS | 125000 | 50 | 40 | 30 | 60.8 | 76000 |
| 27 | ME6 | Ru-SNS | 25000 | 50 | 40 | 30 | 25.0 | 6250 |
| 28 | WE1 | Ru-SNS | 100000 | 50 | 40 | 30 | 96.7 | 96700 |
| 29 | WE1 | Ru-PNN | 50000 | 50 | 40 | 30 | 98.6 | 49300 |
| 30 | WE1 | Ru-PNN | 100000 | 50 | 40 | 30 | 97.3 | 97300 |
| 31 | WE1 | Ru-PNN | 200000 | 50 | 40 | 30 | 62.5 | 125000 |

TABLE 1b

| | | | | | | Hydrogen | | | | |
|---|---|---|---|---|---|---|---|---|---|---|
| | | Substrate | | Loading | Temperature | Pressure | Base | | Conversion, | |
| No. | Substrate | (mmol) | Catalyst | (S/C) | (° C.) | (bar) | (mol %) | Solvent | GC (%) | TON |
| 1 | MM | 10 | Ru-PNN | 50000 | 40 | 30 | 50 | N/A | 99.3 | 49650 |
| 2 | EM | 10 | Ru-SNS | 100000 | 40 | 30 | 50 | N/A | 94.9 | 94900 |
| 3 | ETD | 10 | Ru-SNS | 72000 | 40 | 30 | 30 | N/A | 87.7 | 48672 |
| 4 | ETD | 10 | Ru-SNS | 50000 | 40 | 30 | 50 | N/A | 100.0 | 48650 |
| 5 | MO | 10 | Ru-PNN | 50000 | 40 | 30 | 50 | N/A | 99.3 | 49650 |
| 6 | MO | 10 | Ru-PNN | 50000 | 40 | 30 | 50 | EtOH¹ | 98.1 | 49050 |
| 7 | ED | 17.37 | Ru-SNS | 100000 | 40 | 30 | 50 | N/A | 86.8 | 86800 |

(¹0.1 mL of EtOH was added to the vial prior to the purging sequence)

TABLE 2a

| | | | Catalyst | | Hydrogen | | |
|---|---|---|---|---|---|---|---|
| | | Substrate | loading | Temperature | Pressure | Base | Conversion, |
| Catalyst | No. | (mmol) | (S/C) | (° C.) | (bar) | (mol %) | GC (%) |
| Ru-SNS | 1 | 20 | 100,000/1 | 40 | 30 | 10 | 53.5 |
| | 2 | 20 | 100,000/1 | 40 | 30 | 20 | 85.7 |
| | 3 | 20 | 100,000/1 | 40 | 30 | 30 | 96.8 |
| | 4 | 20 | 100,000/1 | 40 | 30 | 40 | 99.9 |
| Ru-PNN | 5 | 20 | 100,000/1 | 40 | 30 | 10 | 25.4 |
| | 6 | 20 | 100,000/1 | 40 | 30 | 20 | 59.3 |
| | 7 | 20 | 100,000/1 | 40 | 30 | 30 | 97.0 |
| | 8 | 20 | 100,000/1 | 40 | 30 | 40 | 99.9 |

(substrate: ethyl dodecanoate, EE2)

TABLE 2b

| | | | Catalyst | | Hydrogen | | |
|---|---|---|---|---|---|---|---|
| | | Substrate | loading | Temperature | Pressure | Base | Conversion, |
| Catalyst | No. | (mmol) | (S/C) | (° C.) | (bar) | (mol %) | GC (%) |
| Ru-PNN | 1 | 17.37 | 100,000/1 | 40 | 30 | 30 | 70.5 |
| | 2 | 17.37 | 100,000/1 | 40 | 30 | 40 | 83.4 |
| | 3 | 17.37 | 100,000/1 | 40 | 30 | 50 | 92.2 |

(substrate: ethyl decanoate, ED)

TABLE 2c

| | | | Catalyst | | Hydrogen | | |
|---|---|---|---|---|---|---|---|
| | | Substrate | loading | Temperature | Pressure | Base | Conversion, |
| Catalyst | No. | (mmol) | (S/C) | (° C.) | (bar) | (mol %) | GC (%) |
| Ru-SNS | 1 | 17.37 | 50,000/1 | 40 | 12 | 30 | 67.1 |
| | 2 | 17.37 | 50,000/1 | 40 | 12 | 40 | 74.7 |
| | 3 | 17.37 | 50,000/1 | 40 | 12 | 50 | 91.2 |
| Ru-PNN | 6 | 17.37 | 50,000/1 | 40 | 12 | 30 | 73.9 |
| | 7 | 17.37 | 50,000/1 | 40 | 12 | 40 | 81.2 |
| | 8 | 17.37 | 50,000/1 | 40 | 12 | 50 | 88.4 |
| | 9 | 17.37 | 50,000/1 | 40 | 12 | 60 | 94.1 |
| | 10 | 17.37 | 50,000/1 | 40 | 12 | 70 | 95.7 |

(substrate: ethyl decanoate, ED)

TABLE 3

| | | | Catalyst | | Hydrogen | | |
|---|---|---|---|---|---|---|---|
| | | Substrate | loading | Temperature | Pressure | mol % | Conversion, |
| Catalyst | No. | (mmol) | (S/C) | (° C.) | (bar) | base | GC (%) |
| Ru-SNS | 1 | 20 | 0 | 40 | 30 | 50 | 0 |
| | 2 | 20 | 50000/1 | 40 | 30 | 50 | 99.0 |
| | 3 | 20 | 50000/1 | 40 | 30 | 50 | 99.2 |
| | 4 | 20 | 50000/1 | 65 | 30 | 50 | 79.0 |
| | 5 | 20 | 100000/1 | 40 | 30 | 50 | 99.0 |
| | 6 | 20 | 100000/1 | 40 | 30 | 50 | 99.4 |
| | 7 | 20 | 100000/1 | 65 | 30 | 50 | 82.5 |

(substrate: ethyl benzoate, EE1)

TABLE 4

| | | | Catalyst | | Hydrogen | | | | | | |
|---|---|---|---|---|---|---|---|---|---|---|---|
| | | Substrate | Loading | Temperature | Pressure | Base | | | | | C18:1 |
| Catalyst | No. | (mmol) | (S/C) | (° C.) | (bar) | mol % | C16:0 | C18:0 | C18:1 | C18:2 | Conversion |
| Ru-SNS | 1 | 10 | 25,000/1 | 40 | 30 | 50 | 10.1 | 3.6 | 73.9 | 8.1 | 97.7 |
| | 2 | 10 | 50,000/1 | 40 | 30 | 50 | 10.8 | 3 | 75.9 | 8.2 | 100.0 |
| | 3 | 10 | 100,000/1 | 40 | 30 | 50 | 10.2 | 3.6 | 75 | 7.4 | 99.1 |
| | 4 | 10 | 25,000/1 | 65 | 30 | 50 | 10.6 | 3.6 | 74.4 | 7.8 | 98.3 |
| | 5 | 10 | 50,000/1 | 65 | 30 | 50 | 8.4 | 3.1 | 70.5 | 6.9 | 93.2 |
| | 6 | 10 | 100,000/1 | 65 | 30 | 50 | 0 | 0 | 7.31 | 0.96 | 9.7 |
| | 7 | 10 | 25,000/1 | 80 | 30 | 50 | 5.3 | 0.85 | 35.7 | 3.2 | 47.2 |

(substrate: ethyl oleate, EE3)

TABLE 5

| | | | Catalyst | | Hydrogen | | | | | | |
|---|---|---|---|---|---|---|---|---|---|---|---|
| | | Substrate | Loading | Temperature | Pressure | Base | | | | | C18:1 |
| Catalyst | No. | (mmol) | (S/C) | (° C.) | (bar) | mol % | C16:0 | C18:0 | C18:1 | C18:2 | Conversion |
| Ru-PNN | 1 | 10 | 25,000/1 | 40 | 30 | 50 | 9.6 | 3.8 | 73.7 | 8.3 | 97.4 |
| | 2 | 10 | 50,000/1 | 40 | 30 | 50 | 10.6 | 3.6 | 74.3 | 8.1 | 98.2 |
| | 3 | 10 | 100,000/1 | 40 | 30 | 50 | 9.8 | 3.5 | 76.1 | 8.3 | 100.0 |
| | 4 | 10 | 25,000/1 | 65 | 30 | 50 | 10.8 | 3.6 | 74.6 | 7.7 | 98.6 |
| | 5 | 10 | 50,000/1 | 65 | 30 | 50 | 10.6 | 3.9 | 73.8 | 7.8 | 97.6 |
| | 6 | 10 | 100,000/1 | 65 | 30 | 50 | 4.5 | 0.6 | 39 | 4.7 | 51.6 |
| | 7 | 10 | 25,000/1 | 80 | 30 | 50 | 9.7 | 4 | 73.9 | 7.5 | 97.7 |

(substrate: ethyl oleate, EE3)

TABLE 6

| | | | Catalyst | | Hydrogen | | | | | | C18:1 |
|---|---|---|---|---|---|---|---|---|---|---|---|
| | | Substrate | Loading | Temperature | Pressure | Base | | | | | conversion |
| Catalyst | No. | (mmol) | (S/C) | (° C.) | (bar) | mol % | C16:0 | C18:0 | C18:1 | C18:2 | to alcohol |
| Ru-SNS | 1 | 11.79 | 25,000/1 | 40 | 30 | 50 | 4.7 | 4.0 | 80.7 | 10.6 | 99.5 |
| | 2 | 11.79 | 25,000/1 | 50 | 30 | 50 | 3.9 | 3.2 | 79.7 | 9.5 | 98.3 |
| | 3 | 11.79 | 25,000/1 | 60 | 30 | 50 | 4.0 | 3.2 | 79.4 | 9.1 | 97.9 |
| | 4 | 11.79 | 25,000/1 | 70 | 30 | 50 | 3.3 | 4.2 | 56.8 | 7.2 | 70.0 |
| | 5 | 11.79 | 50,000/1 | 40 | 30 | 50 | 3.6 | 3.0 | 82.6 | 10.8 | 100.0 |
| | 6 | 11.79 | 50,000/1 | 50 | 30 | 50 | 4.6 | 4.3 | 71.5 | 9.8 | 88.2 |
| | 7 | 11.79 | 50,000/1 | 60 | 30 | 50 | 2.5 | 2.8 | 45.6 | 5.4 | 56.2 |
| | 8 | 11.79 | 50,000/1 | 70 | 30 | 50 | 1.4 | 1.7 | 31.8 | 4.0 | 39.2 |

(substrate: methyl oleate; reaction duration = 64 h)

TABLE 7

| | | | Catalyst | | Hydrogen | | | | | | Starting | |
|---|---|---|---|---|---|---|---|---|---|---|---|---|
| | | Substrate | Loading | Temperature | Pressure | Base | | | | | Material | C18:1 |
| No. | Catalyst | (mmol) | (S/C) | (° C.) | (bar) | mol % | C16:0 | C18:0 | C18:1 | C18:2 | (%) | Conversion |
| 1 | Ru-SNS | 10 | 50,000/1 | 40 | 10 | 50 | 6.7 | 0 | 72.4 | 7.4 | 1.9 | 97.4 |
| 2 | Ru-PNN | 10 | 50,000/1 | 40 | 10 | 50 | 10 | 0.4 | 66.4 | 9.2 | 3.7 | 94.7 |

(substrate: ethyl oleate, EE3)

TABLE 8a

| | | | Catalyst | | Hydrogen | | Conversion, |
|---|---|---|---|---|---|---|---|
| | | Substrate | loading | Temperature | Pressure | Base | ¹H NMR^{a} |
| Catalyst | No. | (mmol) | (S/C) | (° C.) | (bar) | (mol %) | (%) |
| Ru-PNN | 1 | 20.47 | 50,000/1 | 40 | 30 | 50 | 97.3 |
| | 2 | 20.47 | 50,000/1 | 40 | 30 | 50 | 98.7 |
| | 3 | 20.47 | 50,000/1 | 40 | 30 | 50 | 92.2 |

(substrate: ethyl acetate - first run);
^{a}Conversion calculated from integrals of corresponding product and starting material peaks in the ¹H NMR spectra.

TABLE 8b

| | | | Catalyst | | Hydrogen | Base | | Conversion, |
|---|---|---|---|---|---|---|---|---|
| | | Substrate | loading | Temperature | Pressure | recovered | Base | ¹H NMR^{a} |
| Catalyst | Test | (mmol) | (S/C)^{b} | (° C.) | (bar) | (%) | (mol %) | (%) |
| Ru-PNN | i | 20.47 | 50,000/1 | 40 | 30 | 82.5 | 50 | 92.8 |
| | ii | 20.47 | 50,000/1 | 40 | 30 | 85.8 | 44 | 95.0 |

(substrate: ethyl acetate - second run);
^{a}Conversion calculated from integrals of corresponding product and starting material peaks in the ¹H NMR spectra;
^{b}S/C calculated based upon amount of fresh substrate added prior to second run.

1-28. (canceled)

29. A process for hydrogenation of an ester-containing substrate, comprising treating an ester-containing substrate with a base and a transition metal catalyst in the presence of molecular hydrogen, wherein the base is present in at least 30 mol % based upon the total amount of ester-containing substrate and wherein the substrate/catalyst loading is greater than or equal to 10,000/1.

30. The process as claimed in claim 29, wherein the base is present in at least 35 mol % based upon the total amount of ester-containing substrate.

31. The process as claimed in claim 29, wherein the base is present in at least 40 mol % based upon the total amount of ester-containing substrate.

32. The process as claimed in claim 29, wherein the base is present in at least 45 mol % based upon the total amount of ester-containing substrate.

33. The process as claimed in claim 29, wherein the base is present in at least 50 mol % based upon the total amount of ester-containing substrate.

34. The process as claimed in claim 29, wherein the base is a metal alkoxide.

35. The process as claimed in claim 29, wherein the base is an alkali metal alkoxide.

36. The process as claimed in claim 29, wherein the base is an alkali metal ethoxide selected from lithium ethoxide, sodium ethoxide or potassium ethoxide.

37. The process as claimed in claim 29, wherein the process is carried out in the absence of solvent.

38. The process as claimed in claim 29, wherein the process is carried out in the presence of at least one solvent.

39. The process as claimed in claim 38, wherein the at least one solvent is selected from an alcohol, toluene, THF and Me-THF.

40. The process as claimed in claim 38, wherein said at least one solvent is present in an amount of 10 to 100 vol % based upon the total volume of ester-containing substrate.

41. The process as claimed in claim 29, wherein the process is carried out in the presence of a first solvent and a second solvent.

42. The process as claimed in claim 41, wherein said first solvent is toluene or THF and said second solvent is an alcohol.

43. The process as claimed in claim 41, wherein said first solvent is present in an amount of 10 to 100 vol % based upon the total volume of ester-containing substrate.

44. The process as claimed in claim 41, wherein said second solvent is present in an amount of 1 to 15 vol % based upon the total volume of the ester-containing substrate.

45. The process as claimed in claim 29, wherein the temperature is in the range 20 to 150° C.

46. The process as claimed in claim 29, wherein the pressure is in the range 5 to 100 bar.

47. The process as claimed in claim 29, wherein the substrate/catalyst loading is greater than or equal to 20,000/1.

48. The process as claimed in claim 29, wherein the substrate/catalyst loading is greater than or equal to 30,000/1.

49. The process as claimed in claim 29, wherein the substrate/catalyst loading is greater than or equal to 50,000/1.

50. The process as claimed in claim 29, wherein the substrate/catalyst loading is greater than or equal to 100,000/1.

51. The process as claimed in claim 29, wherein the transition metal catalyst comprises a tridentate ligand.

52. The process as claimed in claim 51, wherein the transition metal catalyst comprises a tridentate ligand having a Formula (I) 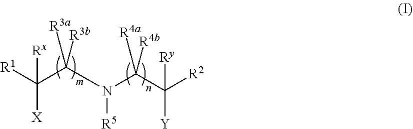
wherein:
X is selected from —SR^{a}, —OR^{a}, —CR^{a}, —NR^{a}R^{b}, —PR^{a}R^{b}, —P(═O)R^{a}R^{b}, —OPR^{a}R^{b}, and —NHPR^{a}R^{b};
R¹ and R^{x} are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl, or R¹ and one of R^{3a} and R^{3b} or R^{x} and one of R^{3a} and R^{3b} together with the atoms to which they are bound, form a ring;
or X is a heteroatom and when taken together with R¹ it forms an optionally substituted heterocycle when R^{X} is absent;
Y is selected from —SR^{a}, —OR^{a}, —CR^{a}, —NR^{a}R^{b}, —PR^{a}R^{b}, —P(═O)R^{a}R^{b}, —OPR^{a}R^{b}, and —NHPR^{a}R^{b};
R² and R^{y} are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl, or R² and one of R^{4a} and R^{4b} or R^{y} and one of R^{4a} and R^{4b} together with the atoms to which they are bound, form a ring;
or Y is a heteroatom and when taken together with R² it forms an optionally substituted heterocycle when R^{Y} is absent;
R^{3a}, R^{3b}, R^{4a} and R^{4b} are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl, or R^{3a} and one of R^{4a} and R^{4b} or R^{3b} and one of R^{4a} and R^{4b}, together with the atoms to which they are bound, form a heterocycle;
R⁵ is selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl;
each m and n is independently 1 or 2; and
R^{a} and R^{b}, if present, are each independently selected from hydrogen, substituted or unsubstituted C₁₋₂₀-alkyl, substituted or unsubstituted C₂₋₂₀-alkenyl, substituted or unsubstituted C₂₋₂₀-alkynyl, substituted or unsubstituted C₁₋₂₀-heteroalkyl, substituted or unsubstituted C₁₋₂₀-alkoxy, substituted or unsubstituted C₃₋₂₀-cycloalkyl, substituted or unsubstituted C₃₋₂₀-cycloalkenyl, substituted or unsubstituted C₂₋₂₀-heterocycloalkyl, substituted or unsubstituted C₆₋₂₀-aryl, and substituted or unsubstituted C₄₋₂₀-heteroaryl; or when X and/or Y is —NR^{a}R^{b}, —PR^{a}R^{b}, —OPR^{a}R^{b}, or —NHPR^{a}R^{b}, R^{a} and R^{b} together with the heteroatom to which they are attached form a heterocycle.

53. The process as claimed in claim 29, wherein the transition metal catalyst has a Formula (II) or Formula (III)
[M(L¹)(L²)_{d}] (II)
[M(L¹)(L²)_{d}]W (III)
wherein:
M is a transition metal;
L¹ is a tridentate ligand wherein the substrate/catalyst loading is greater than or equal to 20,000/1;
L² are ligands which may be the same or different;
d is 1, 2 or 3; and
W is a non-coordinated anionic ligand.

54. The process as claimed in claim 53, wherein M is a transition metal selected from Ru and Os, preferably Ru.

55. The process as claimed in claim 53, wherein each L² is independently selected from —H, —CO, —CN, —P(R′)₃, —As(R′)₃, —CR′, —OR′, —O(C═O)R′, —NR′₂, halogen (e.g. —Cl, —Br, —I), and solvent wherein each R′ is independently selected from substituted or unsubstituted alkyl, substituted or unsubstituted alkenyl, substituted or unsubstituted alkynyl, substituted or unsubstituted heteroalkyl, substituted or unsubstituted alkoxy, substituted or unsubstituted cycloalkyl, substituted or unsubstituted heterocycloalkyl, substituted or unsubstituted aryl, and substituted or unsubstituted heteroaryl.

56. The process as claimed in claim 29, wherein the transition metal catalyst is

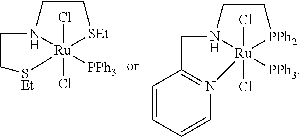

57. The process as claimed in claim 29, wherein the ester-containing substrate is of Formula (X) 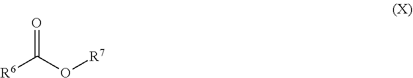
wherein:
R⁶ and R⁷ are independently organic groups having 1-70 carbon atoms; or
R⁶/R⁷ forms a ring structure with the atoms to which they are attached.

58. The process as claimed in claim 29, wherein the ester-containing substrate is a methyl ester or an ethyl ester.

59. The process as claimed in claim 29, which is a batch process, preferably a batch process wherein any excess base is recycled or reused.

60. The process as claimed in claim 29, which is a batch process wherein any excess base is recycled or reused.

61. The process as claimed in claim 29, which is a flow process, preferably a flow process wherein any excess base is recycled or reused.

62. The process as claimed in claim 29, which is a flow process wherein any excess base is recycled or reused.
